(19) United States
(12) Patent Application Publication
(10) Pub. No.: US 2021/0147942 A1
(43) Pub. Date: May 20, 2021

(54) METHOD TO ASSESS SUITABILITY FOR CANCER IMMUNOTHERAPY

(71) Applicant: The Francis Crick Institute Limited, London (GB)

(72) Inventors: Charles Swanton, London (GB); Samra Turajlic, London (GB); Kevin Litchfield, London (GB)

(21) Appl. No.: 16/628,070

(22) PCT Filed: Jul. 4, 2018
(86) PCT No.: PCT/GB2018/051892

§ 371 Date: Jan. 2, 2020

(30) Foreign Application Priority Data
Jul. 5, 2017 (GB) 1710815.0

(51) Int. Cl. C12Q 1/6886; A61K 35/17; C07K 16/28

(52) U.S. Cl. CPC C12Q 1/6886; A61K 35/17; C12Q 2600/106; C07K 16/2827; C07K 16/2818

(57) ABSTRACT

The present invention relates to a method for identifying a subject with cancer who is suitable for treatment with an immune checkpoint intervention, said method comprising analysing in a sample isolated from said subject the expressed frameshift indel mutational burden.

### FIELD OF THE INVENTION

The present invention relates to a method for identifying a subject with cancer who is suitable for treatment with an immune checkpoint intervention. The present invention further relates to methods for predicting whether a subject with cancer will respond to treatment with an immune checkpoint intervention.

### BACKGROUND

Tumour mutation burden (TMB) is associated with response to immunotherapy across multiple tumour types, and therapeutic modalities, including checkpoint inhibitors (CPIs) and cellular based therapies. However, whilst TMB is a clinically relevant biomarker, there are clear opportunities to refine the molecular features associated with response to immunotherapy.

In particular, the primary hypothesis regarding TMB as an immunotherapy biomarker relates to the fact that somatic variants are able to generate tumour specific neoantigens. However, the vast majority of mutations appear to have no immunogenic effect. For example, although hundreds of high affinity neoantigens are predicted in a typical tumour sample, peptide screens routinely detect T cell reactivity against only a few neoantigens per tumour.

There is therefore a need in the art for alternative and improved ways of identifying subjects who will respond to immunotherapies, and for alternative immunotherapy biomarkers. The present invention addresses this need.

The present inventions have found that frame shift insertion/deletions (fs-indels) represent an infrequent (pan-cancer median=4 per tumor) but a highly immunogenic subset of somatic variants. Fs-indels can produce an increased abundance of tumor specific neoantigens with greater mutant-binding specificity. However, fs-indels cause premature termination codons (PTCs) and are susceptible to degradation at the messenger RNA level through the process of non-sense mediated decay (NMD). NMD normally functions as a surveillance pathway to protect eukaryotic cells from the toxic accumulation of truncated proteins. The present inventors have found that a subset of fs-indels escape NMD degradation, which when translated contribute substantially to directing anti-tumour immunity, and therefore represent a biomarker for response to immunotherapy.

### SUMMARY OF THE INVENTION

According to a first aspect the present invention provides a method for identifying a subject with cancer who is suitable for treatment with immunotherapy, said method comprising analysing in a sample isolated from said subject the burden of expressed frameshift indel mutations.

An “indel mutation” as referred to herein refers to an insertion and/or deletion of bases in a nucleotide sequence (e.g. DNA or RNA) of an organism. Typically, the indel mutation occurs in the DNA, preferably the genomic DNA, of an organism. Suitably, the indel mutation occurs in the genomic DNA of a tumour cell in the subject. Suitably, the indel may be an insertion mutation. Suitably, the indel may be a deletion mutation.

Suitably, the indel may be from 1 to 100 bases, for example 1 to 90, 1 to 50, 1 to 23 or 1 to 10 bases.

According to another aspect of the present invention there is provided a method for identifying a subject with cancer who is suitable for treatment with immunotherapy, said method comprising determining the burden of expressed frameshift indel mutations in a sample from said subject, wherein a higher expressed frameshift indel mutational burden in comparison to a reference sample is indicative of response to immunotherapy.

In a further aspect the present invention provides a method for predicting or determining the prognosis of a subject with cancer or predicting survival of a subject with cancer, the method comprising determining the burden of expressed frameshift indel mutations in a sample from said subject, wherein a higher expressed frameshift indel mutational burden is indicative of improved prognosis or improved survival.

The invention further provides a method for predicting or determining whether a type of cancer will respond to treatment with immunotherapy, the method comprising determining the burden of expressed frameshift indel mutations in a sample from said cancer, wherein a higher expressed frameshift indel mutational burden is indicative of response to said treatment.

In a further aspect the present invention provides a method of treating or preventing cancer in a subject, wherein said method comprises the following steps:
(i) identifying a subject with cancer who is suitable for treatment with immunotherapy according to the method of the present invention; and
(ii) treating said subject with immunotherapy.

In another aspect the present invention provides a method of treating or preventing cancer in a subject which comprises the step of administering an immunotherapy to a subject, which subject has been identified as suitable for treatment with immunotherapy using the method of the present invention.

The invention further provides an immunotherapy for use in a method of treatment or prevention of cancer in a subject, the method comprising:
(i) identifying a subject with cancer who is suitable for treatment with an immunotherapy using a method according to the present invention; and
(ii) treating said subject with an immunotherapy.

The invention further provides an immunotherapy for use in treating or preventing cancer in a subject, which subject has been identified as suitable for treatment with immunotherapy using a method according to the present invention.

The present invention therefore addresses a need in the art for new, alternative and/or more effective ways of treating and preventing cancer.

### DESCRIPTION OF THE FIGURES

FIG. 1: (a) Kidney cancers have the highest pan-can indel proportion. Plotted is the proportion of mutations which are indels (i.e. #indels/(#indels+#SNVs), across 19 solid tumour types form TCGA. The last two boxplots are additional independent renal cell carcinoma replication datasets. Statistical association is calculated based on the KIRC cohort compared to all other non-kidney TCGA samples. (b) Kidney cancers have the highest pan-can indel count. Plotted is the absolute count of indel mutations across 19 solid tumour types form TCGA. The last two boxplots are additional independent renal cell carcinoma replication datasets. Statistical association is calculated based on the KIRC cohort compared to all other non-kidney TCGA samples.

FIG. 2: Recurrent genes with frameshift indel neo-antigens, across the all patients in TCGA pan-cancer cohort. Plotted on the X-axis are the number of unique samples containing a frameshift indel neoantigen, and on the Y-axis are the number of unique neo-antigens (i.e. each mutation can generate multiple neo-antigens). Marked are genes either mutated in >30 samples or with >80 neo-antigens.

FIG. 3: Tumour specific neoantigen counts by cancer type. The first panel plots the count of snv derived neo-antigens, second panel is the count of frameshift indel derived neo-antigens, third is the count of mutant only neoantigen binders, fourth is the proportion of neoantigens derived from SNVs/Indels, fifth is the proportion of neo-antigens where mutant allele only binds and last are pie charts presenting the proportion of samples with more or less than 5 mutant only neoantigen binders. The first 3 panels are ordered by median value, from lowest (left) to highest (right). Panels four and five are ordered the same as panel three.

FIG. 4: (a) Non-synonymous SNV mutation burden (first), in-frame indel burden (second) and frameshift indel burden (third) are split by response to checkpoint inhibitor therapy across Hugo et al., Snyder et al., and Van Allen et al. melanoma cohorts. (b) Checkpoint inhibitor patient response rates based on non-synonymous SNV mutation burden (top), in-frame indel burden (middle) and frameshift indel burden (bottom). Patients are split into high (upper quartile) and low (bottom 3 quartiles) groups for each measure. Analysis presented for Hugo et al., Snyder et al., and Van Allen et al. melanoma cohorts.

FIG. 5: Immune gene signatures were compared in ccRCCpatients based on i) frameshift indel neoantigen count (fs-indel-NeoAtgs), ii) in-frame indel mutation count (if-indel-mutations) and iii) nonsynonymousSNV neoantigen count (ns-snv-NeoAtg). Left: Percentage change in median signature expression (FPKM-Upper Quartile normalised) is shown, between high and low groups, for i), ii) and iii). Several pathways were found to be exclusively up-regulated in the high fs-indel-NeoAtggroup. Right: Correlation analysis within the high fs-indel-NeoAtggroup demonstrated the CD8+ T Cell signature was strong correlated with both MHC Class I antigen presentation genes and Cytolytic activity.

FIG. 6: Non-synonymous SNV mutation burden (first), in-frame indel burden (second), frameshift indel burden (third) and clonal frameshift indel burden (fourth) are split by response to checkpoint inhibitor therapy in the Snyder et al., melanoma cohort.

FIG. 7: Panel A shows an overview of study design and methodological approach. The left hand side of the panel shows a fs-indel triggered premature termination codon, which falls in a middle exon of the gene, a position associated with efficient non-sense mediated decay (NMD). The right hand side of the panel shows a fs-indel triggered premature termination codon, which falls in the last exon of the gene, a position associated with bypassing NMD. Panel B shows the odds ratio (OR), between expressed fs-indels and non-expressed fs-indels, for falling into either first, middle, penultimate or last exon positions. Odds ratios and associated p-values were calculated using Fishers Exact Test. Coloring is used arbitrarily to distinguish groups. Error bars denote 95% confidence intervals of OR estimates. Panel C shows variant allele frequencies for expressed fs-indels by exon group position. Kruskal-Wallis test was used to test for a difference in distribution between groups. Panel D shows protein expression levels for non-expressed, versus expressed, fs-indel mutations. Two-sided Mann Whitney U test was used to assess for a difference between groups.

FIG. 8: Panel A shows three melanoma checkpoint inhibitor (CPI) treated cohorts, split into groups based on “no-clinical benefit” or “clinical benefit” to therapy. Three metrics are displayed per cohort: (top row) TMB non-synonymous SNV count, (middle row) frameshift indel count and (bottom row) NMD-escape mutation count. In the first column is the Van Allen et al. anti-CTLA4 cohort, middle column is the Snyder et al. et al. anti-CTLA4 cohort, and the last column is the Hugo et al. anti-PD1 cohort. Far right are meta-analysis p-values, for each metric across the three cohorts, showing the association with clinical benefit from CPI treatment. Two-sided Mann Whitney U test was used to assess for a difference between groups. Meta-analysis of results across cohorts was conducted using the Fisher method of combining P values from independent tests. Panel B shows the % of patient with clinical benefit from CPI therapy, for patients with =>1 NMD-escape mutation and zero NMD-escape mutations. Panel C shows the same three metrics, compared in an adoptive cell therapy treated cohort.

FIG. 9: Shows the exonic positions of fs-indels, experimentally tested for T cell reactivity in personalized vaccine and CPI studies, which were found to either be a) T cell reactive (left hand column) or b) T cell non-reactive (right hand column). Where the fs-indel mutation fell into an exonic position (first, penultimate or last) associated with NMD-escape the transcript was colored dark blue; where the fs-indel fell in an exonic position (middle) associated with NMD-competence the transcript was coloured light blue. In grey line bars the overall proportion of fs-indels falling into an NMD-escape exon position, for T cell reactive and T cell non-reactive groups, is shown. P-value is calculated using a Fisher's Exact Test.

FIG. 10: Panel A shows selection analysis for fs-indels, as benchmarked against functionally equivalent SNV stop-gain mutations. The odds ratio for a fs-indel (compared to SNV stop-gains), to fall into each exon position group is shown. Odds ratios and associated p-values were calculated using Fishers Exact Test. Coloring is used arbitrarily to distinguish groups. Error bars denote 95% confidence intervals of OR estimates. Panel B shows overall survival Kaplan-Meir plots are shown for TCGA SKCM (left) and MSI (right) cohorts. Overall survival analysis was conducted using a Cox proportional hazards model.

FIG. 11: Data shows three melanoma checkpoint inhibitor (CPI) treated cohorts, split into groups based on “no-clinical benefit” (light blue) or “clinical benefit” (dark blue) to therapy, with expressed nsSNV mutation count (detected using allele specific RNAseq) tested for association. In the first column is the Van Allen et al. anti-CTLA4 cohort, middle column is the Snyder et al. et al. anti-CTLA4 cohort, and the last column is the Hugo et al. anti-PD1 cohort.

### DETAILED DESCRIPTION

The present invention is predicated upon the surprising finding that the burden of expressed frameshift indel mutations of a cancer is particularly associated with the response of the subject to immunotherapies such as immune checkpoint intervention or cell therapies. In particular, the present invention is based on the surprising finding that the indel mutational burden—especially the expressed frameshift indel mutational burden—of a cancer is particularly associated with the response of the subject to immune checkpoint intervention or cell therapies compared to other types of mutation, for example single nucleotide variants.

Without wishing to be bound by theory, the present inventors consider that this improved responsiveness to immunotherapy may be provided because indel mutations, particularly expressed frameshift indel mutations, result in the presentation of highly distinct and differential ‘non-self’ peptides by MHC class I molecules compared to other types of mutations (e.g. SNVs). In addition, indel mutations—particularly frameshift mutations—generate an increased number of neoantigens per mutation compared to SNV mutations. These highly distinct non-self peptides provide mutant-specific MHC binding which are recognized by T cells with high affinity TCRs which are present in the subject even after thymic selection and deletion. Accordingly, administration of a checkpoint intervention to the subject releases these high affinity T cells to target an effective T cell mediated immune response against the tumour.

“Indel mutational burden”, as used herein, may refer to “indel mutation number” and/or “indel mutation proportion”.

A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell (e.g. a non-cancer cell) from the same individual and/or the presentation of ‘non-self’ peptides by MHC class I molecules expressed by the tumour cell.

Indel mutations may be identified by Exome sequencing, RNA-seq, whole genome sequencing and/or targeted gene panel sequencing and or routine Sanger sequencing of single genes. Suitable methods are known in the art.

Descriptions of Exome sequencing and RNA-seq are provided by Boa et al. (Cancer Informatics. 2014; 13(Suppl 2):67-82.) and Ares et al. (Cold Spring Harb Protoc. 2014 Nov. 3; 2014(11):1139-48); respectively. Descriptions of targeted gene panel sequencing can be found in, for example, Kammermeier et al. (J Med Genet. 2014 November; 51(11):748-55) and Yap K L et al. (Clin Cancer Res. 2014. 20:6605). See also Meyerson et al., Nat Rev. Genetics, 2010 and Mardis, Annu Rev Anal Chem, 2013. Targeted gene sequencing panels are also commercially available (e.g. as summarised by Biocompare ((http://www.biocompare.com/Editorial-Articles/161194-Build-Your-Own-Gene-Panels-with-These-Custom-NGS-Targeting-Tools/)).

Suitable sequencing methods include, but are not limited to, high throughput sequencing techniques such as Next Generation Sequencing (Illumina, Roche Sequencer, Life Technologies SOLID™), Single Molecule Real Time Sequencing (Pacific Biosciences), True Single Molecule Sequencing (Helicos), or sequencing methods using no light emitting technologies but other physical methods to detect the sequencing reaction or the sequencing product, like Ion Torrent (Life Technologies).

Sequence alignment to identify indels in DNA and/or RNA from a tumour sample compared to DNA and/or RNA from a non-tumour sample may be performed using methods which are known in the art. For example, nucleotide differences compared to a reference sample may be performed using the method as described in the present examples and by Koboldt D C, Zhang Q, Larson D E, Shen D, McLellan M D, Lin L, et al. VarScan 2: somatic mutation and copy number alteration discovery in cancer by exome sequencing. Genome research. 2012; 22(3):568-76.

Nucleotide differences compared to a reference sample may be performed using the methods described in the present Examples. Suitably, the reference sample may be the germline DNA and/or RNA sequence.

In a preferred embodiment, the indel mutation is a frameshift indel mutation. Such frameshift indel mutations generate a novel open-reading frame which is typically highly distinct from the polypeptide encoded by the non-mutated DNA/RNA in a corresponding healthy cell in the subject.

Frameshift mutations typically introduce premature termination codons (PTCs) into the open reading frame and the resultant mRNAs are targeted for nonsense mediated decay (NMD). The present inventors have determined that distinct open-reading frames generated by frameshift indel mutations are able to escape NMD and undergo productive translation to generate polypeptide sequences. Without wishing to be bound by theory, indel frameshift mutations which are not typically targeted for NMD, and will thus generate peptides which can be presented by MHC class I molecules in tumour cells, may be particularly indicative of responsiveness to checkpoint intervention as they provide an effective target for T cell mediated immune responses.

Suitably, the present methods may comprise identifying indel frameshift mutations which are or are not targeted for NMD.

As used herein, the term “expressed indel” is intended to be equivalent to an indel that escapes NMD (and is therefore expressed). As such, an “expressed frameshift indel” is equivalent to a frameshift indel which has escaped NMD.

A high indel mutational burden is defined herein.

### Sample

Isolation of biopsies and samples from tumours is common practice in the art and may be performed according to any suitable method, and such methods will be known to one skilled in the art.

The sample may be a tumour sample, blood sample or tissue sample.

In certain embodiments that sample is a tumour-associated body fluid or tissue.

The sample may be a blood sample. The sample may contain a blood fraction (e.g a serum sample or a plasma sample) or may be whole blood. Techniques for collecting samples from a subject are well known in the art.

Suitably, the sample may be circulating tumour DNA, circulating tumour cells or exosomes comprising tumour DNA. The circulating tumour DNA, circulating tumour cells or exosomes comprising tumour DNA may be isolated from a blood sample obtained from the subject using methods which are known in the art.

Tumour samples and non-cancerous tissue samples can be obtained according to any method known in the art. For example, tumour and non-cancerous samples can be obtained from cancer patients that have undergone resection, or they can be obtained by extraction using a hypodermic needle, by microdissection, or by laser capture. Control (non-cancerous) samples can be obtained, for example, from a cadaveric donor or from a healthy donor.

ctDNA and circulating tumour cells may be isolated from blood samples according to e.g. Nature. 2017 Apr. 26; 545(7655):446-451 or Nat Med. 2017 January; 23(1):114-119.

DNA and/or RNA suitable for downstream sequencing can be isolated from a sample using methods which are known in the art. For example DNA and/or RNA isolation may be performed using phenol-based extraction. Phenol-based reagents contain a combination of denaturants and RNase inhibitors for cell and tissue disruption and subsequent separation of DNA or RNA from contaminants. For example, extraction procedures such as those using DNAzol™, TRIZOL™ or TRI REAGEN™ may be used. DNA and/or RNA may further be isolated using solid phase extraction methods (e.g. spin columns) such as PureLink™ Genomic DNA Mini Kit or QIAGEN RNeasy™ methods. Isolated RNA may be converted to cDNA for downstream sequencing using methods which are known in the art (RT-PCR).

### Subject Suitable for Treatment

In one aspect, the invention provides a method for identifying a subject with cancer who is suitable for treatment with immunotherapy, said method comprising analysing in a sample isolated from said subject the burden of expressed frameshift indel mutations.

As used herein, the term “suitable for treatment” may refer to a subject who is more likely to respond to treatment with immunotherapy, or who is a candidate for treatment with immunotherapy. A subject suitable for treatment may be more likely to respond to said treatment than a subject who is determined not to be suitable using the present invention. A subject who is determined to be suitable for treatment according to the present invention may demonstrate a durable clinical benefit (DCB), which may be defined as a partial response or stable disease lasting for at least 6 months, in response to treatment with immunotherapy.

The number of expressed frameshift indel mutations identified or predicted in the cancer cells obtained from the subject may be compared to one or more pre-determined thresholds. Using such thresholds, subjects may be stratified into categories which are indicative of the degree of response to treatment.

A threshold may be determined in relation to a reference cohort of cancer patients. The cohort may comprise at least 10, 25, 50, 75, 100, 150, 200, 250, 500 or more cancer patients. The cohort may be any cancer cohort. Alternatively the patients may all have the relevant or specific cancer type of the subject in question.

The invention further provides a method for identifying a subject with cancer who is suitable for treatment with immunotherapy, said method comprising determining the burden of expressed frameshift indel mutations in a sample from said subject, wherein a higher expressed frameshift indel mutational burden in comparison to a reference sample is indicative of response to an immunotherapy.

As defined herein, expressed frameshift indel mutational burden may refer to the number of expressed frameshift indel mutations and/or the proportion of indel mutations relative to the total number of mutations.

Suitably, expressed frameshift indel mutational burden may refer to the number of expressed frameshift indel mutations. A “high” or “higher” number of expressed frameshift indel mutations may mean a number greater than the median number of expressed frameshift indel mutations predicted in a reference cohort of cancer patients, such as the minimum number of expressed frameshift indel mutations predicted to be in the upper quartile of the reference cohort.

In another embodiment, a “high” or “higher” number of expressed frameshift indel mutations may be defined as at least 5, 6, 7, 8, 9, 10, 12, 15, or 20 expressed frameshift indel mutations.

Suitably, a “high” or “higher” number of expressed frameshift indel mutational burden may be defined as the contribution of expressed frameshift indel mutations as a proportion of the total mutational count (expressed frameshift indel proportion). Suitably, the expressed frameshift indel proportion may be provided by calculating the number of expressed frameshift indel mutations as a fraction of the total number of mutations.

Suitably, the total number of mutations may be defined as the number of the expressed frameshift indel mutations+the number of SNV mutations. As such, in certain embodiments the expressed frameshift indel proportion may be provided by calculating the number of expressed frameshift indel mutations as a fraction of the total number of expressed frameshift indel mutations+SNV mutations (i.e. number of expressed frameshift indel mutations/number of expressed frameshift indel mutations+SNV mutations).

Suitably, a “high” or “higher” proportion of expressed frameshift indel mutations is greater than the median proportion of expressed frameshift indel mutations determined or predicted in a reference cohort of cancer patients, such as the minimum proportion of expressed frameshift indel mutations determined or predicted to be in the upper quartile of the reference cohort.

In another embodiment, a “high” or “higher” proportion of expressed frameshift indel mutations may be defined as least about 0.06, 0.07, 0.08, 0.09, 0.10, 0.12, 0.15, 0.20, 0.25 or 0.30 of the total number of mutations.

A skilled person will appreciate that references to “high” or “higher” number of expressed frameshift indel mutations may be context specific, and could carry out the appropriate analysis accordingly.

As above, the expressed frameshift indel mutational burden may be determined within the context of a cohort of subjects, either with any cancer or with the relevant/specific cancer. Accordingly, the expressed frameshift indel mutational burden may be determined by applying methods discussed above to a reference cohort. A “high” or “higher” number of expressed frameshift indel mutations may therefore correspond to a number greater than the median number of expressed frameshift indel mutations predicted in a reference cohort of cancer patients, such as the minimum number of expressed frameshift indel mutations predicted to be in the upper quartile of the reference cohort. A “high” or “higher” proportion of expressed frameshift indel mutations may correspond to a proportion greater than the median proportion of expressed frameshift indel mutations predicted in a reference cohort of cancer patients, such as the minimum proportion of expressed frameshift indel mutations predicted to be in the upper quartile of the reference cohort.

Suitably, the present methods may comprise determining both the number of expressed frameshift indel mutations and the proportion of expressed frameshift indel mutations. The number and/or proportion of expressed frameshift indel mutations may be analysed by methods known in the art, e.g. as described in the present Examples.

### Immunotherapy

“Immunotherapy” describes treatments which use the subject's own immune system to fight cancer. It works by aiding the immune system recognise and attack cancer cells.

In one aspect of the present invention as described herein the immunotherapy is immune checkpoint intervention.

Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage. However, whilst immune checkpoints are critical for modulating immune responses in healthy tissues, in the context of cancerous tissues, immune checkpoints can assist a tumour in evading host immune responses that would otherwise work towards eradicating the tumour.

Thus, tumours may co-opt certain immune-checkpoint pathways as a major mechanism of immune resistance, particularly against T cells that are specific for tumour antigens. However, as many of the immune checkpoints are initiated by ligand-receptor interactions, they can be readily blocked by antibodies or modulated by recombinant forms of ligands or receptors. Such interventions have formed the basis of a new line of therapeutic attack against cancers. Cytotoxic T-lymphocyte-associated antigen 4 (CTLA4) antibodies were the first of this class of immunotherapeutics to achieve US Food and Drug Administration (FDA) approval, and a number of other therapeutics have followed.

Whilst immune checkpoint inhibitors are proving to be a useful tool in the ongoing fight against cancer, not all patients respond to such treatments. The present invention facilitates improved identification of patients who will respond to immune checkpoint intervention.

The methods according to the invention as described may further comprise the step of administering an immune checkpoint intervention to a subject who has been identified as suitable for treatment with an immune checkpoint intervention.

Accordingly, the present invention also provides a method of treating or preventing cancer in a subject:
(a) wherein said method comprises:
   (i) identifying a subject with cancer who is suitable for treatment with an immune checkpoint intervention by the method according to present invention;
   (ii) treating said subject with an immune checkpoint intervention;
(b) wherein the subject has been determined to have a higher expressed frameshift indel mutational burden in comparison to a reference sample; or
(c) which subject has been identified as suitable for treatment with an immune checkpoint intervention by the method according to the present invention.

As defined herein “treatment” refers to reducing, alleviating or eliminating one or more symptoms of the disease, disorder or infection which is being treated, relative to the symptoms prior to treatment.

“Prevention” (or prophylaxis) refers to delaying or preventing the onset of the symptoms of the disease, disorder or infection. Prevention may be absolute (such that no disease occurs) or may be effective only in some individuals or for a limited amount of time.

As used herein, “immune checkpoint intervention” may refer to any therapy which interacts with or modulates a signalling interaction or signalling cascade (either at an extracellular or intracellular level) in order to increase/enhance immune cell activity (in particular T cell activity). For example the immune checkpoint intervention may prevent, reduce or minimize the inhibition of immune cell activity (in particular T cell activity). The immune checkpoint intervention may increase immune cell activity (in particular T cell activity) by increasing co-stimulatory signalling.

Suitably, the “immune checkpoint intervention” may be a therapy which interacts with or modulates an immune checkpoint inhibitor molecule. In such embodiments, an immune checkpoint intervention may also be referred to herein as a “checkpoint blockade therapy”, “checkpoint modulator” or “checkpoint inhibitor”.

Immune checkpoint inhibitor molecules are known in the art and include, by way of example, CTLA-4, PD-1, PD-1, Lag-3, Tim-3, TIGIT and BTLA. By “inhibitor” is meant any means to prevent inhibition of T cell activity by, for example, these pathways. This can be achieved by antibodies or molecules that block receptor ligand interaction, inhibitors of intracellular signalling pathways, and compounds preventing the expression of immune checkpoint molecules on the T cell surface.

Checkpoint inhibitors include, but are not limited to, CTLA-4 inhibitors, PD-1 inhibitors, PD-L1 inhibitors, Lag-3 inhibitors, Tim-3 inhibitors, TIGIT inhibitors and BTLA inhibitors, for example. Examples of interventions which may increase immune cell activity include, but are not limited to, co-stimulatory antibodies which deliver positive signals through immune-regulatory receptors including but not limited to ICOS, CD137, CD27 OX-40 and GITR.

Examples of suitable immune checkpoint interventions which prevent, reduce or minimize the inhibition of immune cell activity include pembrolizumab, nivolumab, atezolizumab, durvalumab, avelumab, tremelimumab and ipilimumab.

In one aspect of the invention as described herein the immunotherapy is cell therapy, for example adoptive cell therapy. In one aspect the cell therapy is T cell therapy.

Adoptive cell therapy is the transfer of cells into a patient for the purpose of transferring immune functionality and other characteristics with the cells. The cells are most commonly immune-derived, for example T cells, and can be autologous or allogeneic. If allogenic, they are typically HLA matched. Generally, in cancer immunotherapy, T cells are extracted from the patient, optionally genetically modified, and cultured in vitro and returned to the same patient. Transfer of autologous cells rather than allogeneic cells minimizes graft versus host disease issues. Methods for carrying out adoptive cell therapy are known in the art.

T cells transferred with ACT may be CARTs. Chimeric antigen receptor (CAR) modified T cells (CARTs) have great potential in selectively targeting specific cell types, and utilizing the immune system surveillance capacity and potent self-expanding cytotoxic mechanisms against tumor cells with exquisite specificity. This technology provides a method to target neoplastic cells with the specificity of monoclonal antibody variable region fragments, and to affect cell death with the cytotoxicity of effector T cell function. For example, the antigen receptor can be a scFv or any other monoclonal antibody domain. In some embodiments, the antigen receptor can also be any ligand that binds to the target cell, for example, the binding domain of a protein that naturally associates with cell membrane proteins.

The methods according to the invention as described may further comprise the step of administering a cell therapy to a subject who has been identified as suitable for treatment with an immunotherapy.

Accordingly, the present invention also provides a method of treating or preventing cancer in a subject:
(a) wherein said method comprises:
   (i) identifying a subject with cancer who is suitable for treatment with an immunotherapy by the method according to present invention;
   (ii) treating said subject with cell therapy;
(b) wherein the subject has been determined to have a higher expressed frameshift indel mutational burden in comparison to a reference sample; or
(c) which subject has been identified as suitable for treatment with an immunotherapy by the method according to the present invention.

In one aspect of the invention as described herein, the subject has pre-invasive disease, or is a subject who has had their primary disease resected who might require or benefit from adjuvant therapy.

Treatment using the methods of the present invention may also encompass targeting circulating tumour cells and/or metastases derived from the tumour.

The methods and uses for treating cancer according to the present invention may be performed in combination with additional cancer therapies. In particular, the immune checkpoint interventions according to the present invention may be administered in combination with co-stimulatory antibodies, chemotherapy and/or radiotherapy, targeted therapy or monoclonal antibody therapy.

### Method of Predicting Immunotherapy Treatment Outcome

In a further aspect, the present invention provides a method for predicting or determining whether a subject with cancer will respond to treatment with immunotherapy, the method comprising determining the expressed frameshift indel mutational burden in a sample which has been isolated from said subject.

In view of the surprising findings presented in the present Examples, one skilled in the art would appreciate in the context of the present invention that subjects with a high or higher expressed frameshift indel mutational burden, for example within a cohort of subjects or within a range identified using a number of different subjects or cohorts, may have improved survival relative to subjects with a lower expressed frameshift indel mutational burden.

A reference value for the expressed frameshift indel mutational burden could be determined using the methods provided herein.

The expressed frameshift indel mutational burden may be the expressed frameshift indel mutational number or expressed frameshift indel mutation proportion as defined herein.

Said method may involve determining the expressed frameshift indel mutational burden predicted in a cohort of cancer subjects and either
(i) determining the median number and/or proportion of expressed frameshift indel mutations predicted in that cohort; wherein that median number is the reference value; or
(ii) determining the minimum number and/or proportion of expressed frameshift indel mutations predicted to be in the upper quartile of that cohort, wherein that minimum number and/or proportion is the reference value.

Such a “median number” or “minimum number to be in the upper quartile” could be determined in any cancer cohort per se, or alternatively in the relevant/specific cancer types.

Suitably, a “high” or “higher” number of expressed frameshift indel mutations may be defined as least 3, 4, 5, 6, 7, 8, 9, 10, 12, 15, or 20 indel mutations.

Suitably, a “high” or “higher” proportion of expressed frameshift indel mutations may be defined as least about 0.06, 0.07, 0.08, 0.09, 0.10, 0.12, 0.15, 0.20, 0.25 or 0.30 of the total mutations.

One skilled in the art would appreciate that references to “high” or “higher” expressed frameshift indel mutational burden may be context specific, and could carry out the appropriate analysis accordingly.

As such, the present invention also provides a method for predicting or determining whether a subject with cancer will respond to treatment with immunotherapy, comprising determining the expressed frameshift indel mutational burden in one or more cancer cells from the subject, wherein a higher expressed frameshift indel mutational burden, for example relative to a cohort as discussed above, is indicative of response to treatment or improved survival. In a preferred embodiment the cancer is kidney cancer (renal cell) or melanoma.

### Tumour Suppressors

In one aspect, the expressed frameshift indel mutation may be in a tumour suppressor gene.

A tumour suppressor gene may be defined as a gene that protects a cell from developing to a tumour/cancer cell. Mutations which cause a loss or reduction in function of the protein encoded by a tumour suppressor gene can therefore contribute to the cell progressing to cancer, usually in combination with other genetic changes. Tumour suppressor genes may be grouped into categories including caretaker genes, gatekeeper genes, and landscaper genes.

Proteins encoded by tumour suppressor genes typically have a damping or repressive effect on the regulation of the cell cycle and/or promote apoptosis.

Examples of tumour suppressor genes include, but are not limited to, retinoblastoma (RB), TP53, ARID1A, PTEN, MLL2/MLL3, APC, VHL, CD95, ST5, YPEL3, ST7, ST14 and genes encoding components of the SWI/SNF chromatin remodelling complex.

Thus the present methods may comprise determining the expressed frameshift indel mutational burden in tumour suppressor genes.

### Neoantigens

Suitably, the indel mutation generates a neoantigen. The indel mutation according to the invention as described herein may generate an expressed frameshift neoantigen.

A neoantigen is a tumour-specific antigen which arises as a consequence of a mutation within a cancer cell. Thus, a neoantigen is not expressed by healthy (i.e. non-tumour cells). As described herein, a neoantigen may be processed to generate distinct peptides which can be recognised by T cells when presented in the context of MHC molecules.

Suitably, the expressed frameshift indel mutation generates a clonal neoantigen.

As such, a “clonal” neoantigen is a neoantigen which is expressed effectively throughout a tumour and encoded within essentially every tumour cell. A “branch” or “sub-clonal” neoantigen’ is a neoantigen which is expressed in a subset or a proportion of cells or regions in a tumour.

‘Present throughout a tumour,’ expressed effectively throughout a tumour and ‘encoded within essentially every tumour cell’ may mean that the clonal neoantigen is expressed in all regions of the tumour from which samples are analysed.

It will be appreciated that a determination that a mutation is ‘encoded within essentially every tumour cell’ refers to a statistical calculation and is therefore subject to statistical analysis and thresholds.

Likewise, a determination that a clonal neoantigen is ‘expressed effectively throughout a tumour’ refers to a statistical calculation and is therefore subject to statistical analysis and thresholds.

Expressed effectively in essentially every tumour cell or essentially all tumour cells means that the mutation is present in all tumour cells analysed in a sample, as determined using appropriate statistical methods.

By way of the example, the cancer cell fraction (CCF), describing the proportion of cancer cells that harbour a mutation may be used to determine whether mutations are clonal or sub-clonal. For example, the cancer cell fraction may be determined by integrating variant allele 30 frequencies with copy numbers and purity estimates as described by Landau et al. (Cell. 2013 Feb. 14; 152(4):714-26).

Suitably, CCF values may be calculated for all mutations identified within each and every tumour region analysed. If only one region is used (i.e. only a single sample), only one set of CCF values will be obtained. This will provide information as to which mutations are present in all tumour cells within that tumour region, and will thereby provide an indication if the mutation is truncal or branched. All sub clonal mutations (i.e. CCF<1) in a tumour region are determined as branched, whilst clonal mutations with a CCF=1 are determined to be truncal.

As stated, determining a clonal mutation is subject to statistical analysis and threshold. As such, a mutation may be identified as truncal if it is determined to have a CCF 95% confidence interval >=0.75, for example 0.80, 0.85, 0.90, 0.95, 1.00 or >1.00. Conversely, a mutation may be identified as branched if it is determined to have a CCF 95% confidence interval <=0.75, for example 0.70, 0.65, 0.60, 0.55, 0.50, 0.45, 0.40, 0.35, 0.30, 0.25, 0.20, 0.15, 0.10, 0.05, 0.01 in any sample analysed.

It will be appreciated that the accuracy of a method for identifying truncal mutations is increased by identifying clonal mutations for more than one sample isolated from the tumour.

Thus the present methods may comprise determining the expressed frameshift indel mutational burden of clonal neoantigens.

In certain embodiments, the present methods may comprise determining the expressed frameshift indel mutational burden which generated clonal neoantigens from tumour suppressor genes.

### Subject

In a preferred embodiment of the present invention, the subject is a mammal, preferably a cat, dog, horse, donkey, sheep, pig, goat, cow, mouse, rat, rabbit or guinea pig, but most preferably the subject is a human.

### Cancer

Suitably, the cancer may be ovarian cancer, breast cancer, endometrial cancer, kidney cancer (renal cell), lung cancer (small cell, non-small cell and mesothelioma), brain cancer (gliomas, astrocytomas, glioblastomas), melanoma, Merkel cell carcinoma, clear cell renal cell carcinoma (ccRCC), lymphoma, small bowel cancers (duodenal and jejunal), leukemia, pancreatic cancer, hepatobiliary tumours, germ cell cancers, prostate cancer, head and neck cancers, thyroid cancer and sarcomas.

In one embodiment the cancer may have a mutation in a DNA-repair pathway.

In one embodiment, the cancer is melanoma. In one embodiment, the cancer is kidney cancer (renal cell cancer).

In one embodiment the cancer may be selected from melanoma, Merkel cell carcinoma, renal cancer, non-small cell lung cancer (NSCLC), urothelial carcinoma of the bladder (BLAC), head and neck squamous cell carcinoma (HNSC), and microsatellite instability (MSI)-high cancers.

In one embodiment the cancer may be an MSI-high cancer.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation.

The term “protein”, as used herein, includes proteins, polypeptides, and peptides.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Examples.

### EXAMPLES

### Example 1

The pattern of indel mutations on a pan-cancer basis, and their association with anti-tumour immune response and outcome following checkpoint blockade, was determined.

### Results

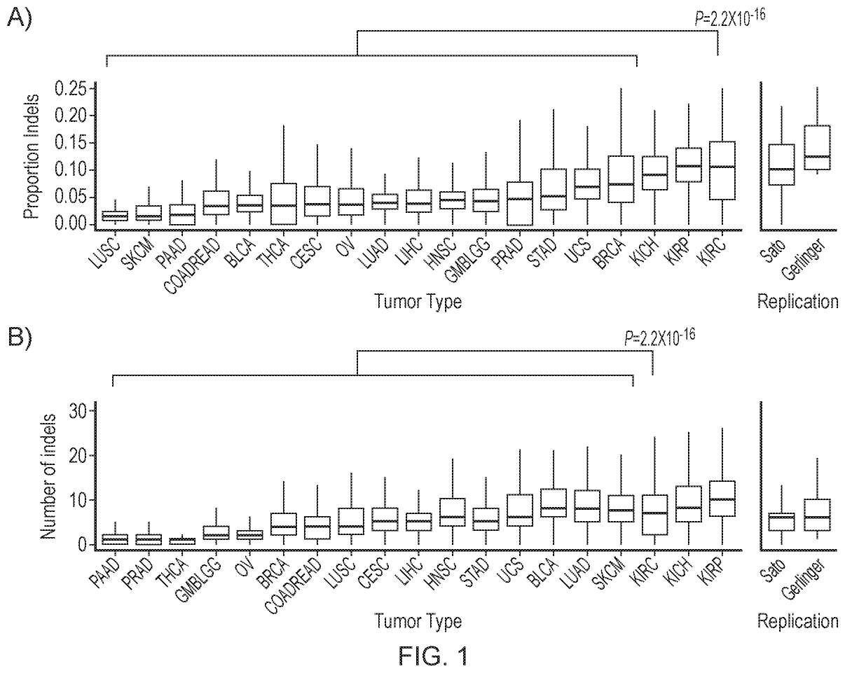

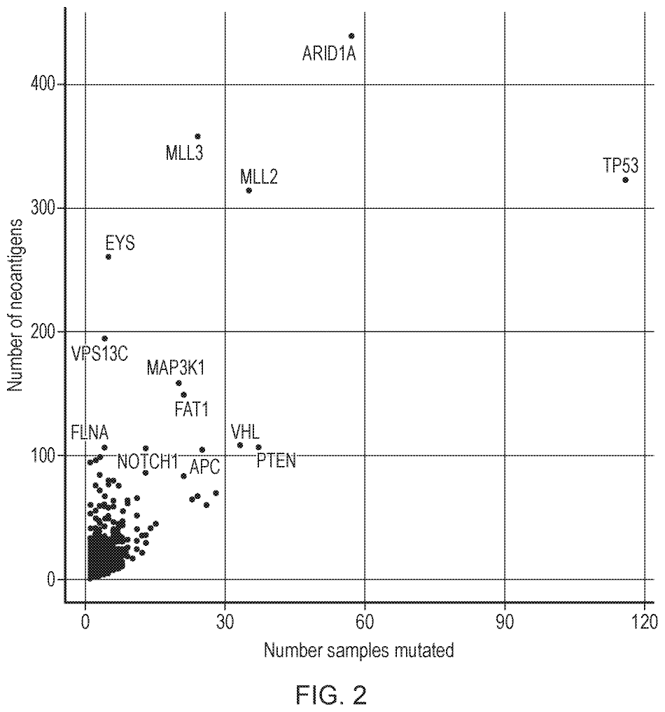

Indel frequencies were compared on a pan-cancer basis, across 19 solid tumour types, utilising 5,777 samples from the cancer genome atlas (TCGA). The contribution of indels was analysed as a proportion of the total mutational count per sample (indel proportion) and the absolute number of indels per sample (indel count) and observed median values of 0.05 and 4 respectively, cohort-wide. Across all tumour types, ccRCC was found to have the highest proportion of coding indels, 0.12 (P=2.2×10⁻¹⁶, FIG. 2), a 2.4-fold increased as compared to the pan-cancer average. This result was replicated in two further independent studies, with median observed indel proportions of 0.10 and 0.12, respectively (1, 2) (FIG. 1). Papillary renal cell carcinoma (pRCC) and chromophobe renal cell carcinoma (chrRCC) had the second/third highest indel proportion, suggesting a possible tissue specific mutational process contributing the acquisition of indels in renal cancers. pRCC, chrRCC and ccRCC also had the highest absolute indel count across all tumour types, with a median indel number of 10, 8, and 7, respectively. ccRCC is characterised by loss of function (LoF) mutations in one or more tumour suppressor genes: VHL, PBRM1, SETD2, BAP1 and KDMSC (11), which can be inactivated by nsSNV or indel mutations. To exclude the possibility these hallmark mutations were distorting the results, ccRCC indel proportion was recalculated excluding VHL, PBRM1, SETD2, BAP1 and KDMSC; the revised indel proportion remained at 0.12. Utilising previously published multiregion whole exome sequencing data from ten ccRCC cases (2) the clonal nature of indel mutations was assessed, revealing 48% of frameshifting indels to be clonal in nature (present in all tumour regions).

For frameshift neo-antigens to contribute to anti-tumour immunity the mutant peptides must be expressed. Frameshifts cause premature termination codons (PTCs) and the resultant mRNAs are targeted for nonsense mediated decay (NMD). Published analyses of germline samples show that PTCs frequently lead to the loss of expression of the variant allele, but that some mutant transcripts escape NMD based on the exact location of the frameshift within a gene (16). Combined analyses of mutational and expression data from over 10,000 cancer samples showed that NMD is triggered with variable efficacy, and even when effective might not alter expression levels due factors such as short mRNA half-life (17). Using the TCGA ccRCC data, the gene expression levels were compared in the samples harbouring a mutation in the given gene, to that in non-mutated samples. This analysis was performed for both indel and SNV mutations, with the latter included as a benchmark comparator. The overall impact of NMD on the expression level of indel mutated genes was estimated to be 14%, markedly below what would be expected under fully operational NMD, pointing to the existence of NMD-evading PTCs.

The potential immunogenicity of nsSNV and indel mutations was determined through analysis of MHC Class I-associated tumour specific neoantigen binding predictions in the pan-cancer TCGA cohort. Across all samples, HLA-specific neoantigen predictions were performed on 335,594 nsSNV mutations, resulting in a total of 214,882 high affinity binders (defined as epitopes with predicted IC50<50 nM), equating to a rate of 0.64 neo-antigens per nsSNV mutation (snv-neo-antigens). In a similar manner predictions were made on 19,849 frameshift indel mutations, resulting in 39,768 high affinity binders with a rate of 2.00 neo-antigens per frameshift mutation (frameshift-neo-antigens). Thus on a per mutation basis, frameshift indels could generate ˜three-fold more high affinity neoantigen binders (Table 1), consistent with the prediction in a recent analyses of a colorectal cancer cohort (18). When both wild type and mutant peptides are predicted to bind central immune tolerance mechanisms may delete cells with the reactive T-cell receptor. Therefore a pan-cancer analyses was repeated, restricting the neo-antigens to mutant specific binders (i.e. where the wild-type peptide is not predicted to bind), and demonstrated that frameshift indels were nine-fold enriched for mutant-allele only binders (Table 1).

TABLE 1
Neo-antigens per variant class

| | | | | No. of | |
|---|---|---|---|---|---|
| | | | | mutant | |
| | No. | No. of | No. | specific | No. |
| Variant | of | neo- | per | neo- | per |
| Class | mutations | antigens* | mutation | antigens** | mutation |
| ns-SNVs | 335,594 | 214,882 | 0.64 | 75,224 | 0.22 |
| fs-Indels | 19,849 | 39,768 | 2.00 | 39,768 | 2.00 |
| Enrichment | | | 3.13 | | 8.94 |

*Strong binders (<50 nM affinity)
**Wildtype allele non-binding (>500 nM affinity)

Of particular interest were genes that are frequently altered via frameshift mutations and with high propensity for MHC binding. In a pan-cancer analysis they were enriched for classic tumour suppressor genes including TP53, ARID1A, PTEN, MLL2/MLL3, APC and VHL (FIG. 2). Collectively the top 15 genes with highest number of frameshifts mutations were mutated in >500 samples (˜10% of the cohort) with >2,400 high affinity neo-antigens predicted. Tumour suppressor genes have been a previously intractable mutational target, but they may be targetable as potent neo-antigens. Furthermore, by virtue of being founder events many alterations in tumour suppressor genes are clonal, present in all cancer cells, rendering them compelling targets for the immune system.

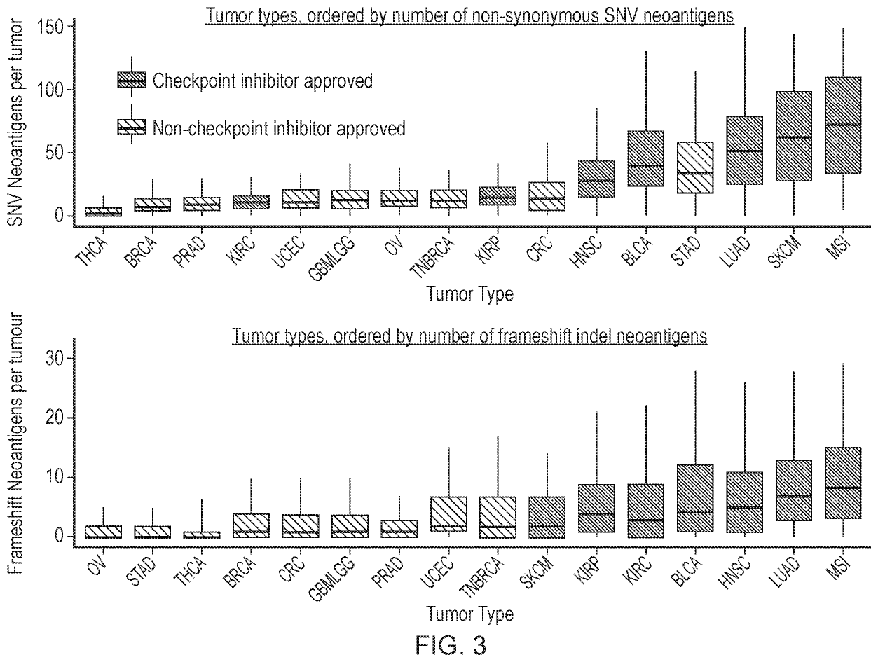

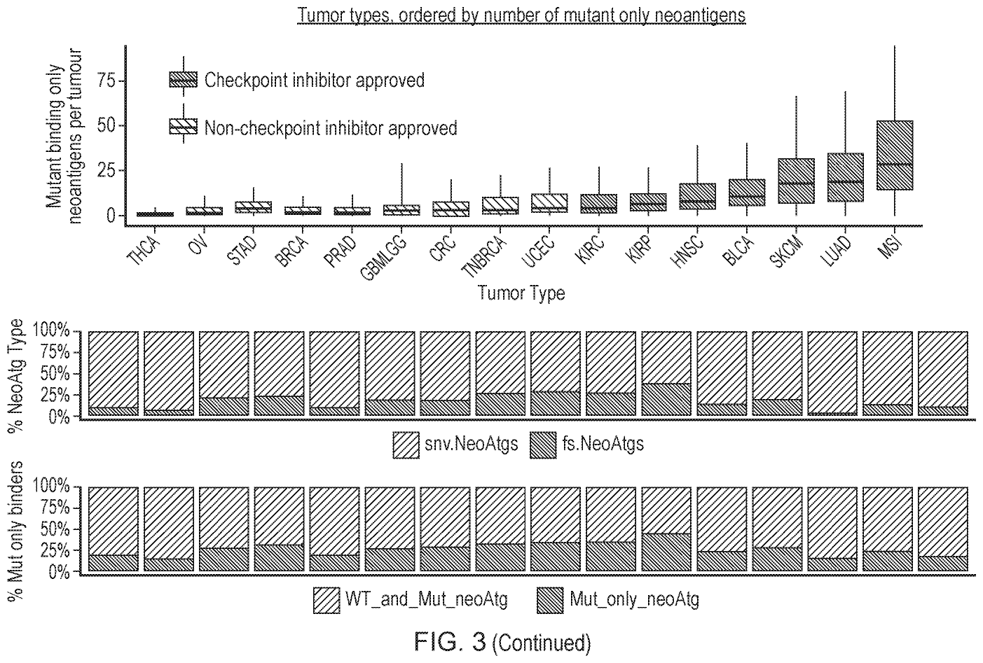

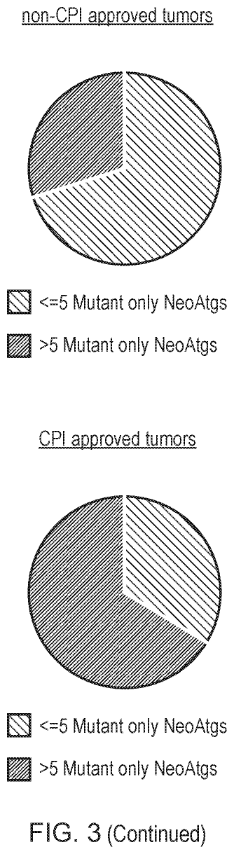

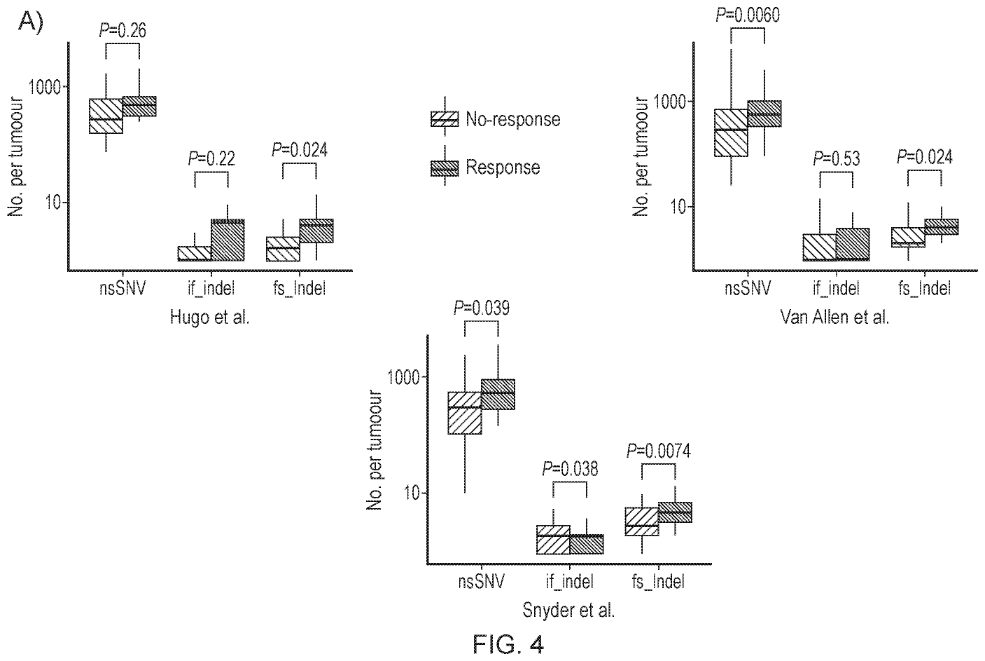

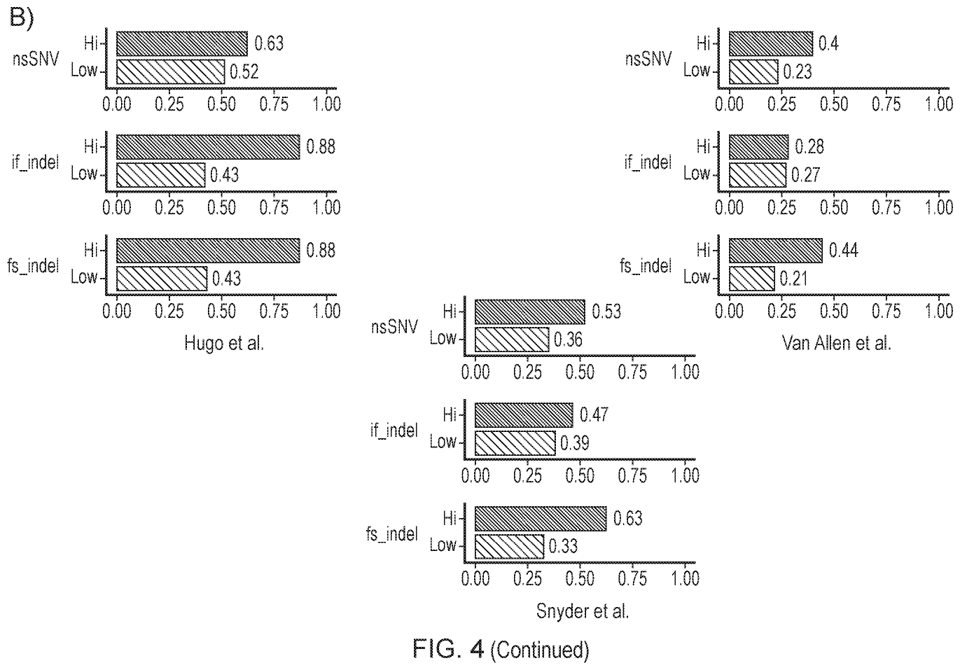

The clinical impact of indel mutations was considered by assessing the relationship between neoantigen enrichment and therapeutic benefit. To date, CPIs have been approved for the treatment of six solid tumour types: melanoma (anti-PD1/CTLA-4), merkel cell carcinoma (anti-PD1), ccRCC (anti-PD1), NSCLC (anti-PD1), BLAC (anti-PD-1) and HNSC (anti-PD1). Consistent with a potential role of frameshifts in the generation of neo-antigens, the CPI approved tumour types were all found to harbour an above average number of frameshift neo-antigens, despite dramatic differences in the total SNV/indel mutational burden, i.e. ccRCC (FIG. 3). Overall, the number of frameshift neo-antigens was considerably higher in the CPI-approved tumour types versus those that are not CPI-approved to date (P=2.2×10⁻¹⁶). The impact of frameshift neo-antigens on CPI efficacy was assessed using exome sequencing results from a recent anti-PD-1 study in melanoma (n=38 patients) (3). Three classes of mutation were defined: (i) non-synonymous SNVs, (ii) in-frame (3n) indels and (iii) frameshift (non-3n) indels, and each tested for an association with response to treatment. While class (i) and (ii) mutations showed a non-significant trend (P=0.26, P=0.22)), class (iii) framehshift indel mutations were significantly associated with anti-PD-1 response, with P=0.02 (FIG. 4a). The upper quartile of patients, with the highest burden of class (iii) frameshift indels, had an 88% response rate (RR) to anti-PD-1 therapy, compared to 43% for the lower three quartiles (FIG. 4b). To confirm the reproducibility of this association CPI response data were obtained from two additional melanoma cohorts with genomic profiling: Snyder et al. (n=62, anti-CTLA-4 treated) (4) and Van Allen et al. (n=100, anti-CTLA-4 treated) (5). The same analyses were conducted in each cohort and frameshift indel burden was significantly associated with CPI response in both additional datasets, with P=0.0074 and P=0.024 respectively (FIG. 4a). An overall meta-analysis across the three cohorts confirmed frameshift indel count to be significantly associated with CPI response (P=3.8×10⁴), and with stronger association than nsSNV count (P=3.5×10³). In addition an improved overall survival was observed in the class (iii) frameshift indel group (Supplementary FIG. 3). Finally, to assess the relationship between frameshift indel load and CPI response in another tumour type, a small cohort of 31 non small cell lung cancer patients treated with anti-PD1 therapy was obtained from Rizvi et al. (6). Although non-significant, a trend of higher frameshift indel load in CPI responders (P=0.2) was observed.

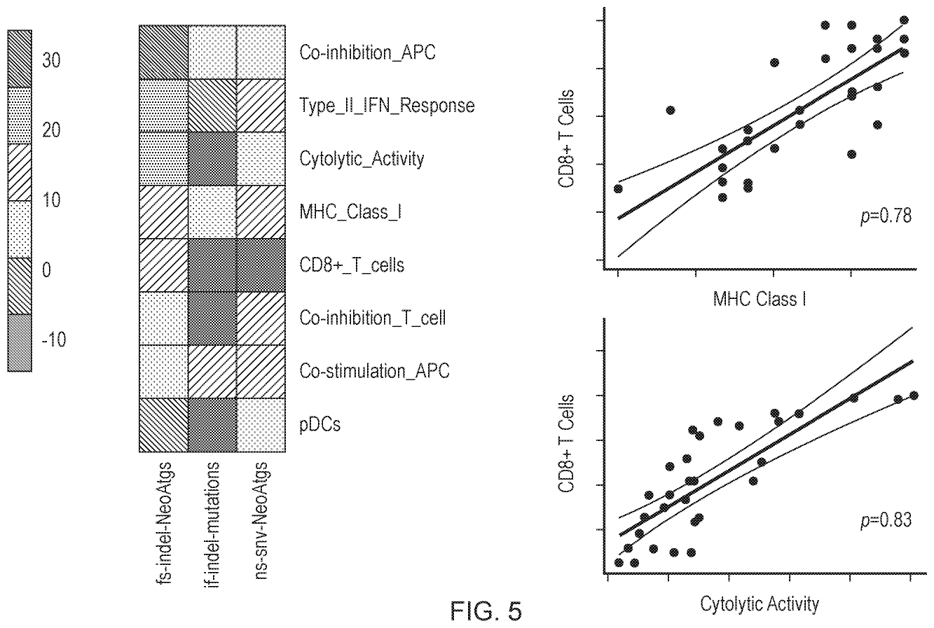

Finally, while genomic data are not available to correlate with CPI response in ccRCC, the relationship between frameshift-neoantigen load and immune responses within the tumour was analysed using RNAseq gene expression data. Patients were split into groups based on the burden of frameshift-neoantignes (high defined as >10 frameshifts/case) versus snv-neoantigens (high defined as >17 nsSNVs/case, with this threshold set to ensure matched patient sample sizes). A high load of frameshift-neo-antigens was associated with up-regulation of immune signatures classically linked to immune activation, including: MHC Class I antigen presentation, CD8+ T cell activation and increased cytolytic activity, a pattern not observed in the high snv-neoantigen group (FIG. 5). Furthermore, correlation analysis within the high frameshift-neoantigen group demonstrated that CD8+ T Cell signature was strongly correlated with both MHC Class I antigen presentation genes and cytolytic activity (p=0.78 and p=0.83 respectively) (FIG. 5).

### Methods

### Study Design and Patients

Pan-cancer somatic mutational data were obtained from the cancer genome atlas (TCGA), for 5,777 available patients who had undergone whole exome sequencing, across 19 different solid tumour types: Bladder urothelial carcinoma (BLCA), Breast invasive carcinoma (BRCA), Cervical and endocervical cancers (CESC), Colorectal adenocarcinoma (COADREAD), Glioma (GMBLGG), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Ovarian serous cystadenocarcinoma (OV), Pancreatic adenocarcinoma (PAAD), Prostate adenocarcinoma (PRAD), Skin Cutaneous Melanoma (SKCM), Stomach adenocarcinoma (STAD), Thyroid carcinoma (THCA) and Uterine Carcinosarcoma (UCS). Patient level mutation annotation files were extracted from the Broad Institute TCGA GDAC Firehose repository (https://gdac.broadinstitute.org/), which had been previously curated by TCGA analysis working group experts to ensure strict quality control. Replication analysis was conducted in two additional ccRCC patient cohorts: i) a whole exome sequencing study of 106 ccRCCs reported by Sato et al (1) ii) a whole exome sequencing study of 10 ccRCCs reported by Gerlinger et al (2). Final post quality control (QC) patient level mutation annotation files were obtained for each study.

In order to test for an association between non-synonymous SNVs/indel loads and patient response to checkpoint inhibitor (CPI) therapy further four patient cohorts were utilised. The first dataset consisted of 38 melanoma patients treated with anti-PD-1 therapy, as reported by Hugo et al. (3). Final post-QC mutation annotation files and clinical outcome data were obtained, and 32 patients were retained for analysis after excluding cases where DNA had been extracted from patient derived cell lines and patients where tissue samples were obtained after CPI therapy. This later exclusion was of particular importance, given the fact CPI therapy itself is likely to alter mutational frequencies through possible elimination of immunogenic tumour clones. The second CPI cohort comprised 62 melanoma patients treated with anti-CTLA-4 therapy, as reported by Snyder et al. (4). All patients samples were taken pre-CPI treatment from fresh snap frozen tumour tissue, so accordingly all 62 cases were retained for analysis. The third CPI cohort comprised 100 melanoma patients treated with anti-CTLA-4 therapy, as reported by Van Allen et al. (5), again all patients were eligible for inclusion using the same criteria as above. The final CPI cohort comprised 31 non small cell lung cancer patients treated with anti-PD1 therapy, as reported by Rizvi et al. (6), again all patients were eligible for inclusion. For the Snyder et al., Van Allen et al. and Rizvi et al. cohorts, final mutation annotation files including indel mutations were not available, so raw BAM files were obtained and variant calling was conducted using a standardized bioinformatics pipeline as described below.

### Whole Exome Sequencing Variant Calling

BAM files representing both the germline and tumour regions from Snyder et al., Van Allen et al. and Rizvi et al. cohorts were obtained and converted to FASTQ format using picard tools (1.107) SamToFastq. Raw paired end reads (100 bp) in FastQ format were aligned to the full hg19 genomic assembly (including unknown contigs) obtained from GATK bundle 2.8 (7), using bwa mem (bwa-0.7.7) (8). Picard tools v1.107 was used to clean, sort and merge files from the same patent region and to remove duplicate reads (http://broadinstitute.github.io/picard). Picard tools (1.107), GATK (2.8.1) and FastQC (0.10.1) (http://www.bioinformatics.babraham.ac.uk/proects/fastac/) were used to produce quality control metrics. SAMtools mpileup (0.1.19) (9) was used to locate non-reference positions in tumour and germline samples. Bases with a phred score of <20 or reads with a mapping-quality <20 were omitted. BAQ computation was disabled and the coefficient for downgrading mapping quality was set to 50. VarScan2 somatic (v2.3.6) (58) utilized output from SAMtools mpileup in order to identify somatic variants between tumour and matched germline samples. Default parameters were used with the exception of minimum coverage for the germline sample that was set to 10 and minimum variant frequency was changed to 0.01. VarScan2 processSomatic was used to extract the somatic variants. The resulting single nucleotide variant (SNV) calls were filtered for false positives using Varscan2's associated fpfilter.pl script, initially with default settings then repeated with again with min-var-frac=0.02, having first run the data through bam-readcount (0.5.1) (https://github.com/genome/bam-readcount). Only INDEL calls classed as ‘high confidence’ by VarScan2 processSomatic were kept for further analysis, with somatic_p_value scores <5×10⁻⁴. MuTect (1.1.4) (10) was also used to detect SNVs utilising annotation files contained in GATK bundle 2.8. Following completion, variants called by MuTect were filtered according to the filter parameter ‘PASS’.

### Pan-Cancer Insertion/Deletion Analysis

In the pan-cancer cohort SNV and insertion/deletion (indel) mutation counts were computed per case, considering all variant types. Across all 5,777 samples a total of 1,227,075 SNVs and 54,207 indels were observed. Dinucleotide and trinucleotide substitutions were not considered. The metric “indel burden” was simply defined as the absolute indel count per case and “indel proportion” was defined as: #indels/(#indels+#SNVs). The same analysis was repeated in the two ccRCC replication cohorts.

### Non-Sense Mediated Decay Analysis

Non-sense mediated decay (NMD) efficiency was estimated using RNAseq expression data (as measured in TPM), obtained from the TCGA GDAC Firehose repository https://gdac.broadinstitute.org/). The extent of NMD was estimated for all indel and SNV mutations by comparing the mRNA expression level in samples with a mutation to the median mRNA expression level of the same transcript across all other tumour samples where the mutation was absent. Specifically, the mRNA expression level of every mutation-bearing transcript was divided by the median mRNA expression level of that transcript in non-mutated samples, to give an NMD index. The overall NMD index values observed were 0.93 (indels) and 1.00 (SNVs), suggesting an overall 0.07 reduction in expression in indel mutated transcripts. Tumour purity in the KIRC cohort is reported to be 0.54 (11), and assuming constant expression levels in the remaining 0.46 normal cellular content, that would yield an adjusted 0.136 drop in expression in indel mutation bearing cancer cells. Assuming tumour mutations are clonal, of heterozygote genotype, in a diploid genomic region and wild-type allele expression in mutated cancer cells remains constant, a purity adjusted reduction of 0.5 would be expected under a model of fully effective NMD. Hence this data suggests NMD operates with reduced efficiency in the KIRC cohort, however we acknowledge the above assumptions will have some impact. These data are presented as a global approximation of NMD efficiency, utilizing methodology in line with previous publications (12).

### Tumour Specific Neoantigen Analysis

For a subset of patients from the TCGA cohort (n=4,592), tumour specific neoantigen binding affinity prediction data was also available and obtained from Rooney et al. (60). In brief, the 4-digit HLA type for each sample, along with mutations in class I HLA genes, were determined using POLYSOLVER (POLYmorphic loci reSOLVER). Somatic mutations were determined using Mutect (14) and Strelka tools. All possible 9 and 10-mer mutant peptides were computed, based on the detected somatic snv and indel mutation across the cohort. Binding affinities of mutant and corresponding wildtype peptides, relevant to the corresponding POLYSOLVER-inferred HLA alleles, were predicted using NetMHCpan (v2.4). Strong affinity binders were defined as IC50<50 nM. Wildtype allele non-binding was defined as IC50>500 nM. We excluded (from the pan-cancer neoantigen analyses) cancers that are associated with a high level of viral genome integration including cervical (>80% rate of HPV integration), hepatocellular carcinoma (>50% rate of HepB integration), but not HNCC (<15% rate of HPV integration). There was no TCGA dataset available for Merkel cell carcinoma.

### Immune Signatures RNAseq Analysis

Immune gene signature data was obtained from Rooney et al. (15) with gene sets defined as per supplementary table 1. Immune signature scores were calculated as the geometric mean of genes within the set, based on RNAseq Transcripts Per Kilobase Million (TPM) expression levels per sample. Analysis was conducted for ccRCC TCGA (KIRC) patients, where both RNAseq and neoantigen data was available (n=392). A high burden of frameshift indel strong affinity neoantigens was defined as >10 per case (n=32), and the percentage difference in expression was compared between the high indel neoantigen group and all other patients, across each immune signature. Immune signatures with minimal expression (<0.5 TPM) in all groups were excluded. The same analysis was repeated for a high burden of snv derived strong affinity neo-antigens, with a threshold of >17 snv neo-antigens selected in order to size match the high burden groups (equal number of patients, n=32 across all high load groups) across mutational types. The percentage differences in expression were plotted in heatmap format. Correlation analysis was conducted within the high frameshift indel neoantigen group (n=32 ccRCC patients).

### Checkpoint Inhibitor (CPI) Response Analysis

Across the four CPI treated patient cohorts (i) non-synonymous SNV, (ii) all coding indel and (iii) frameshift indel variant counts were tested for an association with patient response to therapy. For each measure (i), (ii) and (iii) high and low groups were defined as the top quartile (high) and bottom-three quartiles (low). The same criteria was used across all four datasets, and the proportion of patients responding to therapy (response rate) in high and low groups was compared. Measures of patient response were defined in each study as follows:

### Snyder et al. (4)

Long-term clinical benefit (LB): (i) radio-graphic evidence of freedom from disease or (ii) evidence of a stable disease or (iii) decreased volume of disease; for more than 6 months.
Lack of long-term clinical benefit (NB): (i) tumour growth on every CT scan after the initiation of treatment (no benefit) or (ii) a clinical benefit lasting 6 months or less (minimal benefit).

### Hugo et al. (3):

Responding tumours: complete response (CR), partial response (PR) and stable disease (SD).
Non-responding tumours: disease progression (PD)

### VanAllen et al. (5):

Clinical Benefit:CR/PR/SD
No Clinical benefit: PD or SD with OS<1 year

### Rizvi et al. (6):

Durable clinical benefit (DCB): PR or SD lasting longer than 6 months
No durable benefit (NDB): PD<6 months from beginning of therapy

### Statistical Analysis

Indel burden and proportion measures were compared between ccRCC and all other non-kidney cancers using a two-sided Mann Whitney test. In the CPI response analysis, non-synonymous SNV, exonic indel and frameshift indel counts were each compared to patient response outcome using a two-sided Mann Whitney test. Meta-analysis of results across the four CPI datasets was conducted using the Fisher method of combining P values from independent tests. Immune signature correlation analysis was conducted using a spearman's rank correlation coefficient. Statistical analyses were carried out using R3.0.2 (http://www.r-project.org/). A P value of 0.05 (two sided) was considered as being statistically significant.

### Clonality

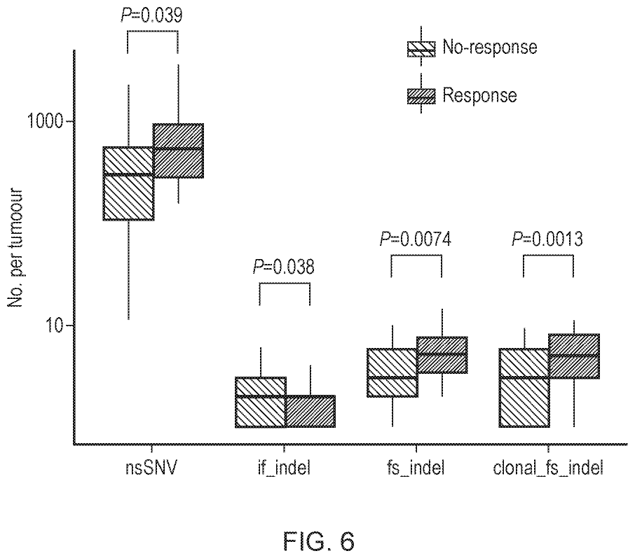

The impact of clonality was additionally assessed, and clonal frameshift indels were found to have a further predictive advantage beyond all frameshift indels (clonal and subclonal). See FIG. 6 in this regard).

### REFERENCES

1. Sato Y, Yoshizato T, Shiraishi Y, Maekawa S, Okuno Y, Kamura T, et al. Integrated molecular analysis of clear-cell renal cell carcinoma. Nat Genet. 2013; 45(8):860-7.
2. Gerlinger M, Horswell S, Larkin J, Rowan A J, Salm M P, Varela I, et al. Genomic architecture and evolution of clear cell renal cell carcinomas defined by multiregion sequencing. Nat Genet 2014; 46(3):225-33.
3. Hugo W, Zaretsky J M, Sun L, Song C, Moreno B H, Hu-Lieskovan S, et al. Genomic and Transcriptomic Features of Response to Anti-PD-1 Therapy in Metastatic Melanoma. Cell. 2017; 168(3):542.
4. Snyder A, Makarov V, Merghoub T, Yuan J, Zaretsky J M, Desrichard A, et al. Genetic basis for clinical response to CTLA-4 blockade in melanoma. N Engl J Med. 2014; 371(23):2189-99.
5. Van Allen E M, Miao D, Schilling B, Shukla S A, Blank C, Zimmer L, et al. Genomic correlates of response to CTLA-4 blockade in metastatic melanoma. Science. 2015; 350(6257):207-11.
6. Rizvi N A, Hellmann M D, Snyder A, Kvistborg P, Makarov V, Havel J J, et al. Cancer immunology. Mutational landscape determines sensitivity to PD-1 blockade in non-small cell lung cancer. Science. 2015; 348(6230):124-8.
7. McKenna A, Hanna M, Banks E, Sivachenko A, Cibulskis K, Kemytsky A, et al. The Genome Analysis Toolkit a MapReduce framework for analyzing next-generation DNA sequencing data. Genome research. 2010; 20(9):1297-303.
8. Li H, Durbin R. Fast and accurate short read alignment with Burrows-Wheeler transform. Bioinformatics. 2009; 25(14):1754-60.
9. Li H, Handsaker B, Wysoker A, Fennell T, Ruan J, Homer N, et al. The Sequence Alignment/Map format and SAMtools. Bioinformatics. 2009; 25(16):2078-9
10. Cibulskis K, Lawrence M S, Carter S L, Sivachenko A, Jaffe D, Sougnez C, et al. Sensitive detection of somatic point mutations in impure and heterogeneous cancer samples. Nature biotechnology. 2013; 31(3):213-9.
11. Cancer Genome Atlas Research N. Comprehensive molecular characterization of clear cell renal cell carcinoma. Nature. 2013; 499(7456):43-9.
12. Lindeboom R G, Supek F, Lehner B. The rules and impact of nonsense-mediated mRNA decay in human cancers. Nat Genet 2016; 48(10):1112-8.
13. Rooney M S, Shukla S A, Wu C J, Getz G, Hacohen N. Molecular and genetic properties of tumours associated with local immune cytolytic activity. Cell. 2015; 160(1-2):48-61.
14. Cibulskis K, Lawrence M S, Carter S L, Sivachenko A, Jaffe D, Sougnez C, et al. Sensitive detection of somatic point mutations in impure and heterogeneous cancer samples. Nature biotechnology. 2013; 31(3):213-9.
15. Jamal-Hanjani M, Wilson G A, McGranahan N, Birkbak N J, Watkins TBK, Veeriah S, et al. Tracking the Evolution of Non-Small-Cell Lung Cancer. N Engl J Med. 2017.
16. Lappalainen T, Sammeth M, Friedlander M R, t Hoen P A, Monlong J, Rivas M A, et al. Transcriptome and genome sequencing uncovers functional variation in humans. Nature. 2013; 501(7468):506-11.
17. Lindeboom R G, Supek F, Lehner B. The rules and impact of nonsense-mediated mRNA decay in human cancers. Nat Genet. 2016; 48(10):1112-8.
18. Giannakis M, Mu X J, Shukla S A, Qian Z R, Cohen O, Nishihara R, et al. Genomic Correlates of Immune-Cell Infiltrates in Colorectal Carcinoma. Cell Rep. 2016; 17(4):1206.

### Example 2

It was determined in Example 1 that fs-indels are associated with improved response to checkpoint inhibitor therapy. The effects of non-sense mediated decay were then investigated.

### Materials and Methods

Study Cohorts Matched DNA/RNA sequencing analysis was conducted in the following cohorts all treated with immunotherapy:
Van Allen et al. (8), an advanced melanoma checkpoint inhibitor (CPI) (anti-CTLA-4) treated cohort. Cases with both RNA sequencing and whole exome (DNA) sequencing data were utilised (n=33).
Snyder et al. (7), an advanced melanoma CPI (anti-CTLA-4) treated cohort. Cases with both RNA sequencing and whole exome (DNA) sequencing data were utilised (n=21).
Hugo et al. (4), an advanced melanoma CPI (anti-PD-1) treated cohort. Cases with both RNA sequencing and whole exome (DNA) sequencing data were utilised (n=24).
Lauss et al. (10), an advanced melanoma adoptive cell therapy treated cohort. Cases with both RNA sequencing and whole exome (DNA) sequencing data were utilised (n=22).
Snyder et al. (18), a metastatic urothelial cancer CPI (anti-PD-L1) treated cohort. Cases with both RNA sequencing and whole exome (DNA) sequencing data were utilised (n=23).

Matched DNA/RNA sequencing analysis was conducted in the following cohorts (not specifically treated with immunotherapy):
Skin cutaneous melanoma (SKCM) tumors, obtained from the cancer genome atlas (TCGA) project. Cases with paired end RNA sequencing data and curated variant calls from TCGA GDAC Firehose (2016_01_28 release) were utilised (n=368).
Microsatellite instable (MSI) tumors, across all histological subtypes from TCGA project. MSI cases IDs were identified based on classification from Cortes-Ciriano et al. (19). Cases with paired end RNA sequencing data and curated variant calls from TCGA GDAC Firehose (2016_01_28 release) were utilised (n=96).

Prediction of NMD-escape features (based on DNA exonic mutation position only, rather than matched DNA/RNA sequencing analysis) was conducted in the following immunotherapy treated cohorts:
Ott et al. (22), an advanced melanoma personalized vaccine treated cohort (n=6 cases).
Rahma et al. (23), a metastatic renal cell carcinoma personalized vaccine treated cohort (n=6 cases).
Le et al. (24), an advanced mismatch repair-deficient cohort, across cancers across 12 different tumor types, treated with anti-PD-1 blockade (n=86 cases, functional neoantigen reactivity T cell work only conducted in n=1 case).

### Whole Exome Sequencing (DNA) Variant Calling

For Van Allen et al. (8), Snyder et al. (7) and Snyder et al. (18) cohorts, we obtained germline/tumor BAM files from the original authors and reverted these back to FASTQ format using Picard tools (version 1.107) SamToFastq. Raw paired-end reads in FastQ format were aligned to the full hg19 genomic assembly (including unknown contigs) obtained from GATK bundle (version 2.8), using bwa mem (bwa-0.7.7). We used Picard tools to clean, sort and to remove duplicate reads. GATK (version 2.8) was used for local indel realignment. We used Picard tools, GATK (version 2.8), and FastQC (version 0.10.1) to produce quality control metrics. SAMtools mpileup (version 0.1.19) was used to locate non-reference positions in tumor and germline samples. Bases with a Phred score of less than 20 or reads with a mapping quality less than 20 were omitted. VarScan2 somatic (version 2.3.6) used output from SAMtools mpileup to identify somatic variants between tumour and matched germline samples. Default parameters were used with the exception of minimum coverage for the germline sample, which was set to 10, and minimum variant frequency was changed to 0.01. VarScan2 processSomatic was used to extract the somatic variants. Single nucleotide variant (SNV) calls were filtered for false positives with the associated fpfilter.pl script in Varscan2, initially with default settings then repeated with min-var-frac=0.02, having first run the data through bam-readcount (version 0.5.1). MuTect (version 1.1.4) was also used to detect SNVs, and results were filtered according to the filter parameter PASS. In final QC filtering, an SNV was considered a true positive if the variant allele frequency (VAF) was greater than 2% and the mutation was called by both VarScan2, with a somatic p-value <=0.01, and MuTect. Alternatively, a frequency of 5% was required if only called in VarScan2, again with a somatic p-value <=0.01. For small scale insertion/deletions (INDELs), only calls classed as high confidence by VarScan2 processSomatic were kept for further analysis, with somatic_p_value scores less than 5×10⁻⁴. Variant annotation was performed using Annovar (version 2016 Feb. 1). Variants in either the first, penultimate or last exon, of the relevant transcript as annotated first (default) by Annovar, were considered to be mutations in exonic positions associated with NMD-escape. Middle exon mutations were considered to be all those not in first, penultimate or last exon positions. For the Hugo et al. (4) cohort, we obtained final post-quality control mutation annotation files generated as previously described (4). Briefly, SNVs were detected using MuTect, VarScan2 and the GATK Unified Genotyper, while INDELs were detected using VarScan2, IndelLocator and GATK-UGF. Mutations that were called by at least two of the three SNV/INDEL callers were retained as high confidence calls. For the Lauss et al. (10) cohort, SNVs and INDELs were called as described previously (10). Briefly, SNVs were detected using the intersection of MuTect and VarScan2 variants, while INDELs were detected using VarScan2 only. For VarScan2, high confidence calls at a VAF greater than 10% were retained.

### Whole Transcriptome Sequencing (RNA) Variant Calling

RNAseq data was obtained in BAM format for all studies, and reverted back to FASTQ format using bam2fastq (v.1.0). Insertion/deletion mutations were called from raw paired end FASTQ files, using mapsplice (v2.2.0), with sequence reads aligned to hg19 genomic assembly (using bowtie pre-built index). Minimum QC thresholds were set to retain variants with =>5 alternative reads, and variant allele frequency=>0.05. Insertions and deletions called in both RNA and DNA sequencing assays were intersected, and designated as expressed indels, with a +/−10 bp padding interval included to allow for minor alignment mismatches. SNVs in RNA sequencing data were called directly from the hg19 realigned BAM files, using Rsamtools to extract read counts per allele for each genomic position where a SNV had already called in DNA sequencing analysis. Similarly, minimum QC thresholds of =>5 alternative reads, and variant allele frequency=>0.05, were utilised and variants passing these thresholds were designated as expressed SNVs.

### Protein Expression Analysis

We retrieved Level 4 (L4) normalized protein expression data for 223 proteins, across n=453 TCGA melanoma/MSI tumors (which overlapped with the TCGA cohorts also analysed via DNA/RNA sequencing) from the cancer proteome atlas (http://tcpaportal.org/tcpa/index.html). We filtered the data to sample/protein combinations which also contained an fs-indel mutation (n=136), as called by DNA sequencing. The dataset was then split into two groups, based on the fs-indel being expressed or not (as measured by RNAseq, using the method detailed above). The two groups were compared using a two-sided Mann Whitney test.

### Outcome Analysis

Across all immunotherapy treated cohorts, measures of patient clinical benefit/no-clinical benefit were kept as consistent with original author's criteria/definitions. For TCGA outcome analysis, overall survival (OS) data was utilized, based on clinical annotation data obtained from TCGA GDAC Firehose repository.

### Selection Analysis

To test for evidence of selection, fs-indel mutations were compared to stop-gain SNV mutations, in the SKCM TCGA cohort (n=368 cases). Stop-gain SNV mutations were utilised a benchmark comparator, due to their likely equivalent functional impact (i.e. loss of function), equivalent treatment by the NMD pathway (i.e. last exon stop-gain SNVs will still escape NMD and cause truncated protein accumulation) but lack of immunogenic potential (i.e. no mutated peptides are generated). Across all SKCM cases n=1,594 fs-indels and n=9,833 stop-gain SNVs were considered. All alterations in each group were annotated for exon position (i.e. first, middle, penultimate or last exon, as defined above). The odds of having an fs-indel in first, middle, penultimate or last exon positions was then benchmarked against the equivalent odds for a stop-gain SNV.

### Statistical Methods

Odds ratios were calculated using Fisher's Exact Test for Count Data, with each exon position group compared to all others. Kruskal-Wallis test was used to test for a difference in distribution between three or more independent groups. Two-sided Mann Whitney U test was used to assess for a difference in distributions between two population groups. Meta-analysis of results across cohorts was conducted using the Fisher method of combining P values from independent tests. Logistic regression was used to assess multiple variables jointly for independent association with binary outcomes. Overall survival analysis was conducted in the SKCM TCGA cohort using a Cox proportional hazards model, with stage, sex and age included as covariates. Overall survival analysis was conducted in the MSI TCGA cohort using a Cox proportional hazards model, with primary disease site included as a covariate. Statistical analysis were carried out using R3.4.4 (http://www.r-troiect.or/). We considered a P value of 0.05 (two sided) as being statistically significant.

### Results

### Detection of NMD-Escape Mutations

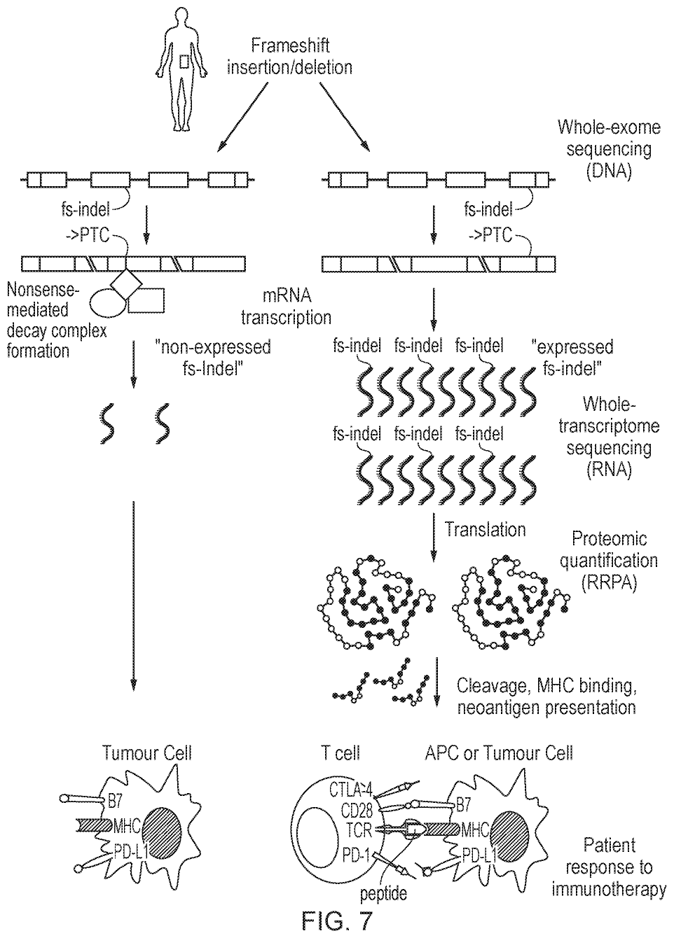

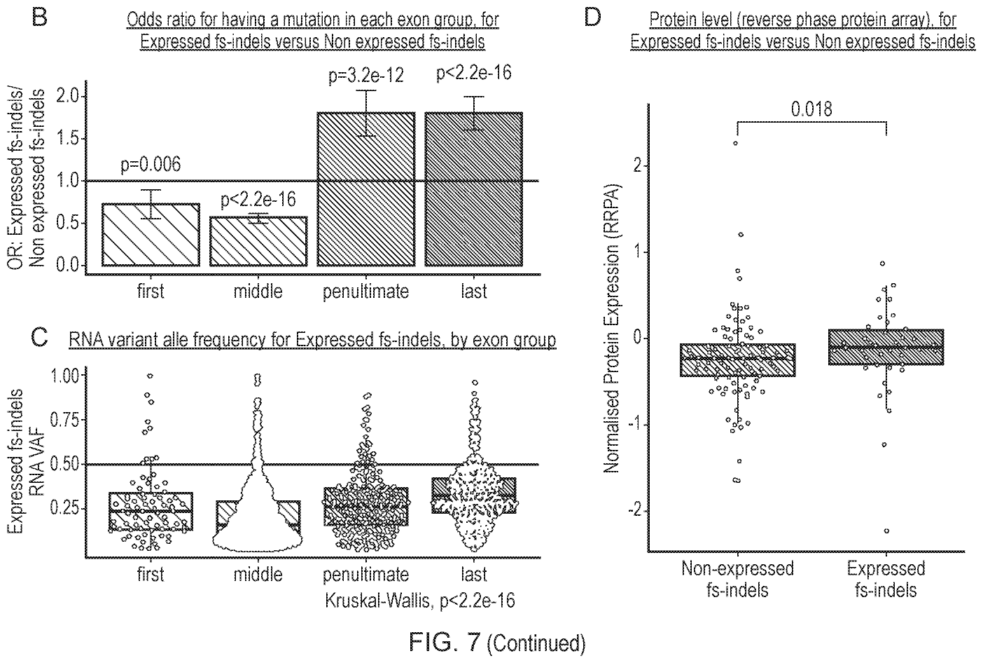

Expressed frameshift indels (fs-indels) were detected using paired DNA and RNA sequencing, with data processed through an allele specific bioinformatics pipeline (FIG. 7A). Across all processed TCGA samples (n=453, see methods for cohort details) a median of 4 fs-indels were detected per tumor (range 0-470), of which mutant allele expression was detected in a median of 1 per tumor (range 0-94). Thus, expressed fs-indel mutations were present at relatively low frequency and abundance. In fact, 49.6% of samples profiled had zero expressed fs-indel mutations detected. Exon positions were annotated for expressed fs-indels (n=1,840), and compared to non-expressed fs-indels (i.e. mutant allele present in DNA, but not in RNA) (n=8,691). Expressed fs-indels were enriched for mutations in penultimate (odds ratio versus non expressed fs-indels=1.80, 95% confidence interval [1.53-2.11], p=3.2×10⁻¹²) and last exon positions (OR=1.80 [1.60-2.04], p<2.2×10⁻¹⁶), while being depleted in middle exon locations (OR=0.56 [0.51-0.62], p<2.2×10⁻¹⁶) (FIG. 7B). These exon positions are consistent with known patterns of NMD-escape, as previously established (14). First exon position mutations were unexpectedly depleted (OR=0.71 [0.55-0.91, p=0.006), however the absolute number of observed mutations in this group was small (only n=80 expressed fs-indels) and a proportion of them (n=21) were >200 nt from the gene start. Next we considered RNA variant allele frequency (VAF) estimates for expressed fs-indels, and found them to be highest for last (median=0.33), penultimate (0.28) and then first (0.26) exon positions, with middle exon alterations having the lowest value (0.19) (FIG. 7C, p<2.2×10⁻¹⁶). Finally, we obtained protein expression data from the cancer proteome atlas (17), for 223 proteins across 453 tumors, which overlapped with the DNA/RNAseq processed cohort. Intersecting samples with both an fs-indel gene mutation(s), and matched protein expression data, we compared the protein levels of expressed (n=40) versus non expressed fs-indels (n=96). Protein abundance was found to be significantly higher for expressed fs-indels (p=0.018, FIG. 7D). Taken collectively, these results suggest that expressed fs-indels are (at least partially) escaping NMD and being translated to the protein level. Expressed fs-indels are here after referred to as NMD-escape, and non-expressed fs-indels as NMD-competent.

NMD-Escape Mutation Burden Associates with Clinical Benefit to Immune Checkpoint Inhibition

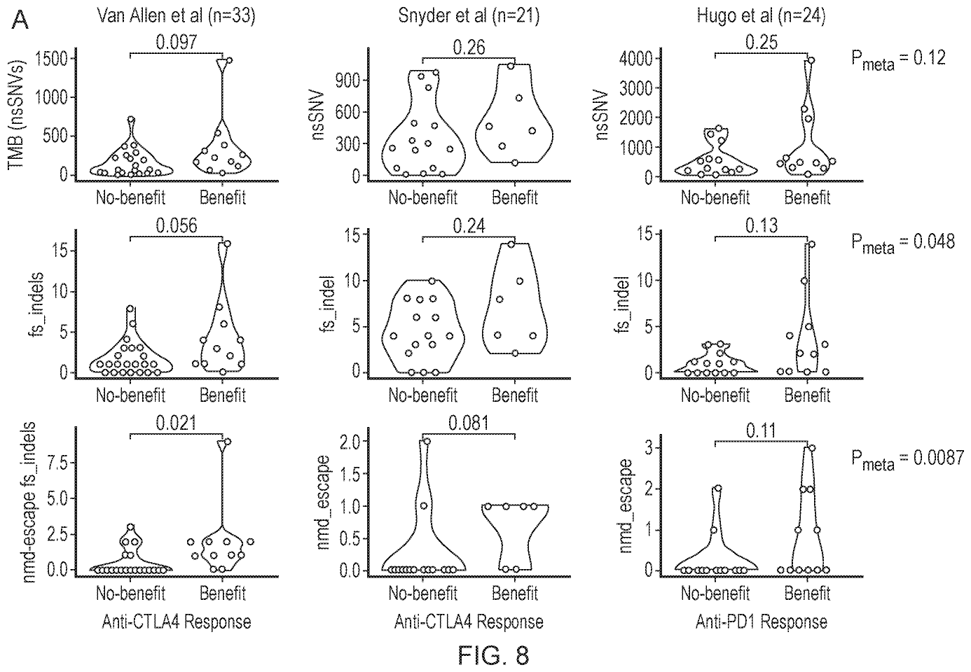

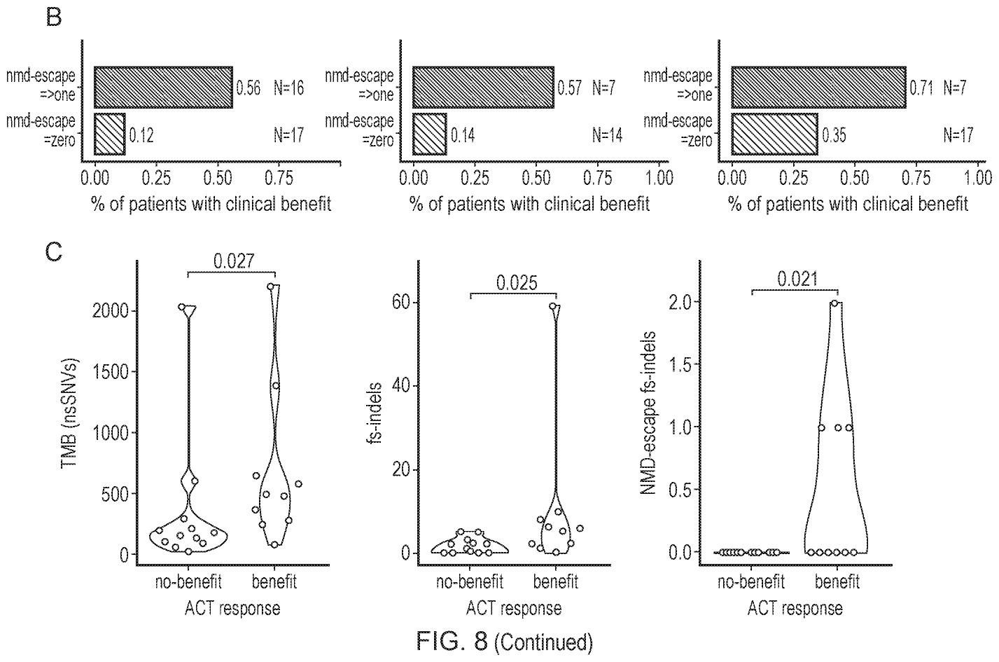

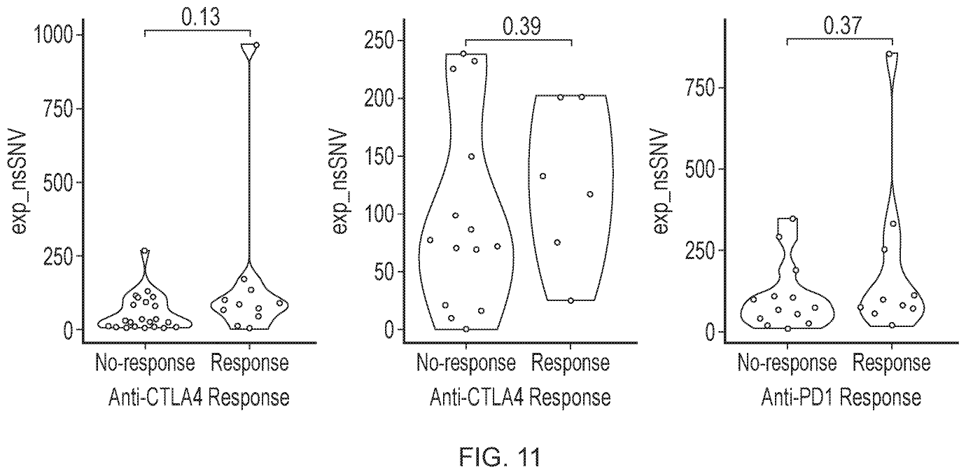

To assess the impact of NMD-escape mutations on anti-tumor immune response, we assessed the association between NMD-escape mutation count and CPI clinical benefit in three independent melanoma cohorts with matched DNA and RNA sequencing data: Van Allen et al. (n=33, anti-CTLA-4 treated), Snyder et al. (n=21, anti-CTLA-4 treated) and Hugo et al. (n=24, anti-PD-1 treated). For each sample, mutation burden was quantified based on the following classifications: i) TMB: all non-synonymous SNVs (nsSNVs), ii) fs-indels, and iii) NMD-escape fs-indels. Each mutation class was tested for an association with clinical benefit (FIG. 8a). In the pooled meta-analysis of the three melanoma cohorts with both WES and RNAseq (total n=78), a trend towards significance was observed for nsSNVs (meta-analysis across all cohorts, Pₘₑₜₐ=0.12) and marginal significance for fs-indels (Pₘₑₜₐ=0.048), while NMD-escape mutation count had the strongest overall association with clinical benefit (Pₘₑₜₐ=0.0087) (FIG. 8a). For clarity, we note sample sizes utilised here are smaller than previously reported, since only a subset of cases had both matched DNA and RNA sequencing data available, and that nsSNV and fs-indel measures are significant in the full datasets. Patients with one or more NMD-escape mutation had higher rates of clinical benefit to immune checkpoint blockade compared to patients with no NMD-escape mutations: 56% versus 12% (Van Allen et al.), 57% versus 14% (Snyder et al.), and 71% versus 35% (Hugo et al.) (FIG. 8b). To ensure the NMD-escape group was not simply reflecting the importance of neoantigen expression in general, we examined expressed nsSNVs detected using allele-specific RNAseq analysis and found that the association with clinical benefit remained non-significant (Pₘₑₜₐ=0.24, FIG. 11). We additionally assessed for evidence of correlation between TMB and nmd-escape metrics, and found only a weak correlation between the two variables (r=0.21, P=0.06, n=78). And in multivariate logistic regression analysis, we tested both variables together in a joint model to assess for independent significance (n=78, study ID was also included as a model term to control for cohort specific factors), and NMD-escape mutation count was found to independently associate with CPI clinical benefit (P=0.032), whereas TMB did not reach independent significance (P=0.25). Finally to investigate a potential association in other tumor types, NMD-escape analysis was conducted in a CPI treated metastatic urothelial cancer cohort (n=23 cases) (18). Previous analysis in this study found that neither TMB, predicted neoantigen load nor expressed neoantigen load, were associated with CPI clinical benefit (18). Similarly, here we found no evidence of an association between NMD-escape count and clinical benefit (P=1.0), possibly due to small sample size, lower mutational load lower in this cohort (TMB=˜0-5 missense SNVs/megabase, as compared to ˜10.0 in a larger recently published cohort (9)), or lower response rates in general in metastatic urothelial cancer. For completeness, the NMD-escape CPI meta-analysis was repeated to include the above bladder data, together with the three melanoma cohorts, and the association remains significant (Pₘₑₜₐ=0.028).

Clinical Benefit to Adoptive Cell Therapy (ACT) Associates with NMD-Escape Mutation Burden

To further investigate the importance of NMD-escape mutations in directing anti-tumor immune response, we analysed matched DNA and RNA sequencing data from patients with melanoma (n=22) treated with adoptive cell therapy (10). TMB ns-SNVs (P=0.027), fs-indels (P=0.025) and NMD-escape count (P=0.021) were all associated with clinical benefit from therapy (FIG. 8c). All patients with NMD-escape count a 1 experienced clinical benefit (n=4, 100%), compared to 33% (6/18) of patients who had no NMD-escape mutations, further highlighting the potential strong immunogenic effect from just a single NMD-escape mutation. As previously reported (10), patients with high nsSNV load (defined as the upper tertile of patients) had improved progression free survival compared to patients with intermediate (middle tertile) or low (bottom tertile) nsSNV count (P=0.0008). We note that of the patients with NMD-escape count a 1, the majority (3 of 4) were in the intermediate (rather than high) tertile nsSNV group, and may have been missed as high likelihood potential responders if TMB alone was used a predictive biomarker. The hazard ratio (HR) per single NMD-escape mutation was 0.28 (95% confidence interval 0.07-1.09), equivalent to approximately 845 nsSNV mutations (HR=0.28 (0.08-0.92)) (Table S1).

TABLE S1
Multivariate analysis

| PFS | Adjusted hazard ratio (95% Cl) | P-value |
|---|---|---|
| TMB (per mutation) | 0.9985 (0.997-0.9999) | 0.038 |
| NMD escape (per mutation) | 0.2812 (0.073-1.0865) | 0.0658 |
| TMB (per 845 mutations) | 0.2813 (0.079-0.919) | |
| NMD escape (per mutation) | 0.2812 (0.073-1.0865) | |

### Table S1

Multivariate progression free survival analysis results are shown for Lauss et al cohort, using a Cox proportional hazards model, with nsSNVs and NMD-escape mutation counts both included in the model as continuous variables. The first table shows the adjusted hazard ratio per single mutation for each measure, and the second table shows the comparable hazard ratio for how many TMB (nsSNVs) mutations are required to equal the same risk reduction as one NMD-escape mutation.

### T Cell Reactivate Neoantigens are Enriched in Genomic Positions Predicted to Escape NMD

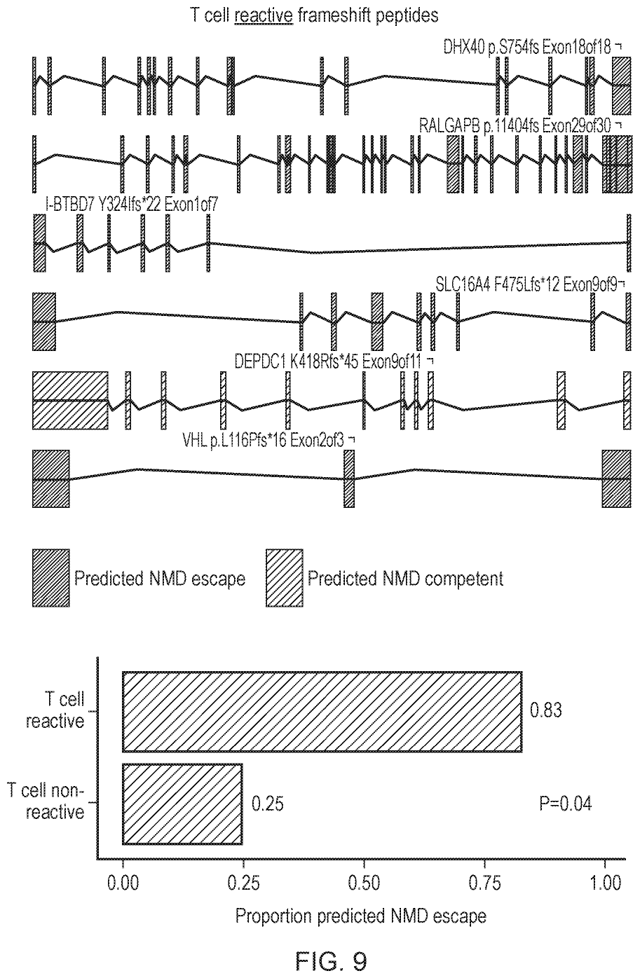

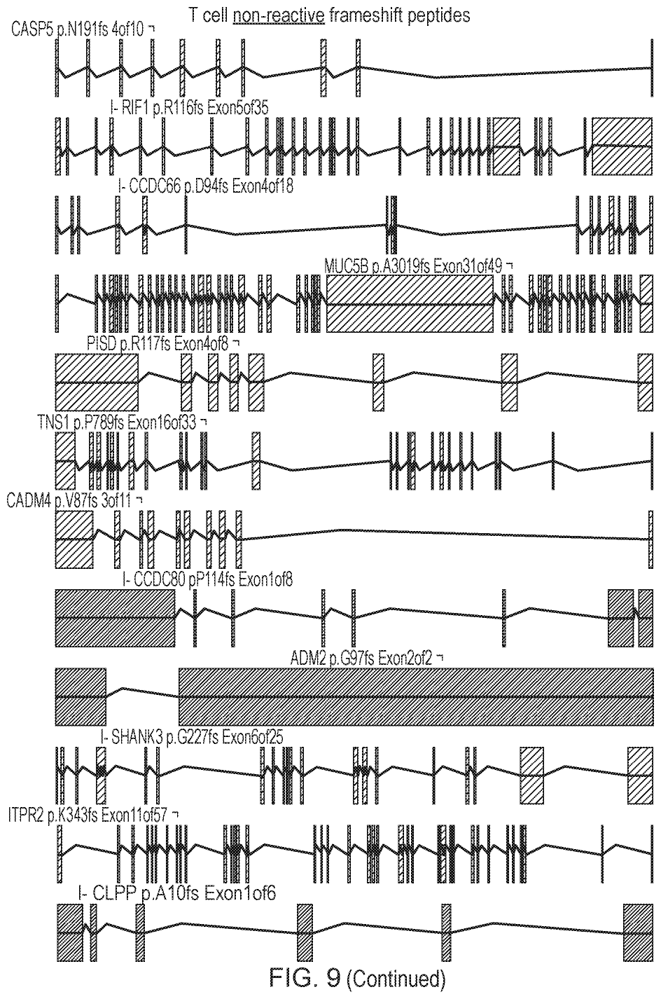

While of translational relevance and clinical utility, biomarker associations do not directly isolate specific neoantigens driving anti-tumor immune response. Accordingly, we obtained data from two anti-tumor personalised vaccine studies and one CPI study in which T cell reactivity against specific neopeptides had been established by functional assay of patient T cells. Across these three studies, six fs-indel derived neoantigens were functionally validated as eliciting T cell reactivity: DHX40 p.S754fs, RALGAPB p.l1404fs, BTBD7 p.Y324fs, SLC16A4 p.F475fs, DEPDCI p.K418fs, and VHL p.L116fs (FIG. 9). Thus, at a proof of concept level, the ability of fs-indels to elicit anti-tumor immune response has been previously established. Across these same studies, 12 fs-indel derived neoantigens had also undergone functional screening, but were found to be T cell non-reactive (FIG. 9). Paired DNA and RNA sequencing data were not available for all these cohorts to determine expression, so annotation of exonic position was used to estimate the likelihood of NMD escape. Within the group of fs-indels shown to be T cell reactive, 5 out of 6 were annotated in exon positions with reduced NMD efficiency (i.e. first, penultimate and last exon), compared to only 3 out of 12 for fs-indel peptides screened but with no T cell reactivity found (FIG. 9). While exceptions are observed (i.e. middle exon position mutations eliciting T cell response, and conversely last exon position mutations failing to generate T cell reactivity), an enrichment is observed with T cell reactive fs-indels more likely to occur in NMD-escape exon positions (OR=12.5 [0.9-780.7], P=0.043) (FIG. 9).

### NMD-Escape Mutations Show Evidence of Negative Selection

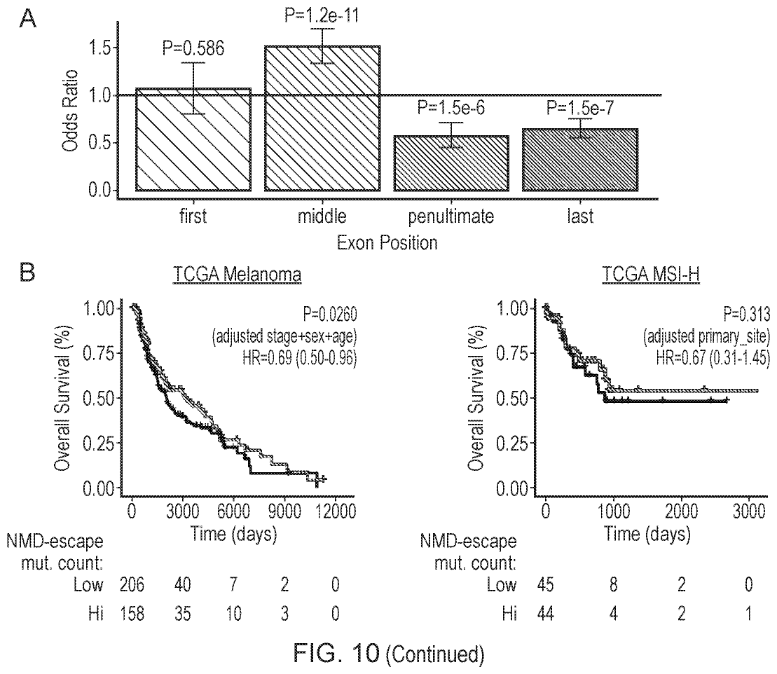

Next, we assessed for evidence of selective pressure against NMD-escape mutations, which may reflect the potential to generate native anti-tumor immunogenicity. In additional to potential immunogenic selective pressure, fs-indels have also previously been reported to be under functional selection (15) due to their loss of protein function effect. To account for this, we used stop-gain SNV mutations as a benchmark comparator, as these variants have equivalent functional impact but no immunogenic potential (i.e. loss of function but no neoantigens generated). Furthermore, the rules of NMD apply equally to both stop-gain SNVs and fs-indels, as both trigger premature termination codons. Using the skin cutaneous melanoma (SKCM) TCGA cohort, we annotated all fs-indel (n=1,594) and stop-gain (n=9,883) mutations for exonic position. Penultimate and last exon alterations were found to be significantly depleted in fs-indels compared to stop-gain events (OR=0.58 [0.46-0.71], P=1.5×10⁻⁵ and OR=0.65 [0.55-0.75], P=1.5×10⁻⁷ respectively) (FIG. 10A). By contrast fs-indel mutations were more likely to occur in middle exon positions (OR=1.51 [1.33-1.68], P=1.2×10⁻¹¹). First exon mutations were not enriched either way, possibly due to small absolute numbers (only n=69 fs-indels were first exon). This data suggests negative selective immune pressure acts against fs-indel mutations in exonic positions likely to escape NMD (e.g. penultimate and last), leading to cancer cells with middle exon fs-indels being more likely to survive immunoediting.

NMD-Escape Mutation Burden is Associated with Improved Overall Survival

Finally, to assess evidence of natural anti-tumor immunogenicity of NMD-escape mutations in melanomas, we examined matched DNA and RNA sequencing data from 368 patients in the TCGA SKCM cohort. Patients with at least one NMD-escape mutation had significantly improved OS (HR=0.69 [0.50-0.96], P=0.03), as compared to those with zero NMD-escape mutations (FIG. 10B). Additionally, using matched DNA and RNA sequencing data from MSI carcinomas (which have high abundance of fs-indel events) identified by Cortes-Ciriano et al. (19) (n=96), a similar but non-significant trend in improved OS was observed among patients with high NMD-escape mutation load (defined as >cohort median value rather than =>1, due to the high level of indel events) (HR=0.67 [0.31-1.45], P=0.313).

The results presented herein show that expressed fs-indels are highly enriched in genomic positions predicted to escape NMD, and have higher protein-level expression (relative to non-expressed fs-indels). Expressed fs-indels (a.k.a. NMD-escape mutations) also significantly associated with clinical benefit from immunotherapy.

NMD-escape mutation count was found to significantly associate with clinical benefit from immunotherapy, across both CPI and ACT modalities, and with a stronger association than either nsSNVs or fs-indels. CPI clinical benefit rates for patients with 2 one NMD-escape mutation were elevated (range across the cohorts analysed=0.56-0.71) compared to patients with zero such events (range 0.12-0.35). Furthermore experimental evidence, analyzed from anti-tumor vaccine and CPI studies, demonstrates T cell reactivity against expressed frameshifted neoepitopes directly in human patients. T cell reactive fs-indel neoantigens were enriched in NMD-escape exon positions (OR=12.5 [0.9-780.7], P=0.043, versus experimentally screened, but T cell non-reactive fs-indels.

### REFERENCES

1. Forde P M, Chaft J E, Smith K N, Anagnostou V, Cottrell T R, Hellmann M D, et al. Neoadjuvant PD-1 Blockade in Resectable Lung Cancer. N Engl J Med. 2018; 378(21):1976-86.
2. Hellmann M D, Callahan M K, Awad M M, Calvo E, Ascierto P A, Atmaca A, et al. Tumor Mutational Burden and Efficacy of Nivolumab Monotherapy and in Combination with Ipilimumab in Small-Cell Lung Cancer. Cancer Cell. 2018; 33(5):853-61 e4.
3. Hellmann M D, Ciuleanu T E, Pluzanski A, Lee J S, Otterson G A, Audigier-Valette C, et al. Nivolumab plus Ipilimumab in Lung Cancer with a High Tumor Mutational Burden. N Engl J Med. 2018; 378(22):2093-104.
4. Hugo W, Zaretsky J M, Sun L, Song C, Moreno B H, Hu-Lieskovan S, et al. Genomic and Transcriptomic Features of Response to Anti-PD-1 Therapy in Metastatic Melanoma. Cell. 2016; 165(1):35-44.
5. Rizvi N A, Hellmann M D, Snyder A, Kvistborg P, Makarov V, Havel J J, et al. Cancer immunology. Mutational landscape determines sensitivity to PD-1 blockade in non-small cell lung cancer. Science. 2015; 348(6230):124-8.
6. Roh W, Chen P L, Reuben A, Spencer C N, Prieto P A, Miller J P, et al. Integrated molecular analysis of tumor biopsies on sequential CTLA-4 and PD-1 blockade reveals markers of response and resistance. Sci Transl Med. 2017; 9(379).
7. Snyder A, Makarov V, Merghoub T, Yuan J, Zaretsky J M, Desrichard A, et al. Genetic basis for clinical response to CTLA-4 blockade in melanoma. N Engl J Med. 2014; 371(23):2189-99.
8. Van Allen E M, Miao D, Schilling B, Shukla S A, Blank C, Zimmer L, et al. Genomic correlates of response to CTLA-4 blockade in metastatic melanoma. Science. 2015; 350(6257):207-11.
9. Mariathasan S, Turley S J, Nickles D, Castiglioni A, Yuen K, Wang Y, et al. TGFbeta attenuates tumour response to P D-L1 blockade by contributing to exclusion of T cells. Nature. 2018; 554(7693):544-8.
10. Lauss M, Donia M, Harbst K, Andersen R, Mitra S, Rosengren F, et al. Mutational and putative neoantigen load predict clinical benefit of adoptive T cell therapy in melanoma. Nat Commun. 2017; 8(1):1738.
11. Robbins P F, Lu Y C, E I-Gamil M, Li Y F, Gross C, Gartner J, et al. Mining exomic sequencing data to identify mutated antigens recognized by adoptively transferred tumor-reactive T cells. Nat Med. 2013; 19(6):747-52.
12. Bassani-Stemberg M, Braunlein E, Kar R, Engleitner T, Sinitcyn P, Audehm S, et al. Direct identification of clinically relevant neoepitopes presented on native human melanoma tissue by mass spectrometry. Nature communications. 2016; 7:13404.
13. Turajlic S, Litchfield K, Xu H, Rosenthal R, McGranahan N, Reading J L, et al. Insertion-and-deletion-derived tumour-specific neoantigens and the immunogenic phenotype: a pan-cancer analysis. Lancet Oncol. 2017; 18(8):1009-21.
14. Lindeboom R G, Supek F, Lehner B. The rules and impact of nonsense-mediated mRNA decay in human cancers. Nat Genet. 2016; 48(10):1112-8.
15. Hu Z, Yau C, Ahmed A A. A pan-cancer genome-wide analysis reveals tumour dependencies by induction of nonsense-mediated decay. Nat Commun. 2017; 8:15943.
16. Maby P, Galon J, Latouche J B. Frameshift mutations, neoantigens and tumor-specific CD8(+) T cells in microsatellite unstable colorectal cancers. Oncoimmunology. 2016; 5(5):e1115943.
17. Li J, Lu Y, Akbani R, Ju Z, Roebuck P L, Liu W, et al. TCPA: a resource for cancer functional proteomics data. Nature methods. 2013; 10(11):1046-7.
18. Snyder A, Nathanson T, Funt S A, Ahuja A, Buros Novik J, Hellmann M D, et al. Contribution of systemic and somatic factors to clinical response and resistance to P D-L1 blockade in urothelial cancer—An exploratory multi-omic analysis. PLoS Med. 2017; 14(5):e1002309.
19. Cortes-Ciriano I, Lee S, Park W Y, Kim T M, Park P J. A molecular portrait of microsatellite instability across multiple cancers. Nat Commun. 2017; 8:15180.
20. Apcher S, Daskalogianni C, Lejeune F, Manoury B, Imhoos G, Heslop L, et al. Major source of antigenic peptides for the MHC class I pathway is produced during the pioneer round of mRNA translation. Proc Nai Acad Sci USA. 2011; 108(28):11572-7.
21. Rock K L, York I A, Saric T, Goldberg A L. Protein degradation and the generation of MHC class I-presented peptides. Adv Immunol. 2002; 80:1-70.
22. Ott P A, Hu Z, Keskin D B, Shukla S A, Sun J, Bozym D J, et al. An immunogenic personal neoantigen vaccine for patients with melanoma. Nature. 2017; 547(7662):217-21.
23. Rahma O E, Ashtar E, Ibrahim R, Toubaji A, Gause B, Herrin V E, et al. A pilot clinical trial testing mutant von Hippel-Lindau peptide as a novel immune therapy in metastatic renal cell carcinoma. Journal of translational medicine. 2010; 8:8.
24. Le D T, Durham J N, Smith K N, Wang H, Bartlett B R, Aulakh L K, et al. Mismatch repair deficiency predicts response of solid tumors to PD-1 blockade. Sciene. 2017; 357(6349):409-13.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for identifying a subject with cancer who is suitable for treatment with immunotherapy, said method comprising analysing in a sample isolated from said subject the burden of expressed frameshift indel mutations.

2. A method for identifying a subject with cancer who is suitable for treatment with immunotherapy, said method comprising determining the burden of expressed frameshift indel mutations in a sample from said subject, wherein a higher expressed frameshift indel mutational burden in comparison to a reference sample is indicative of response to immunotherapy.

3. A method for predicting or determining whether a subject with cancer will respond to treatment with immunotherapy, the method comprising determining the burden of expressed frameshift indel mutations in a sample from said subject, wherein a higher expressed frameshift indel mutational burden is indicative of response to said treatment.

4. A method for predicting or determining whether a type of cancer will respond to treatment with immunotherapy, the method comprising determining the burden of expressed frameshift indel mutations in a sample from said cancer, wherein a higher expressed frameshift indel mutational burden is indicative of response to said treatment.

5. A method of treating or preventing cancer in a subject, wherein said method comprises the following steps:
(i) identifying a subject with cancer who is suitable for treatment with immunotherapy by the method according to claim 1 or 2; and
(ii) treating said subject with an immunotherapy.

6. A method of treating or preventing cancer in a subject which comprises treating a subject with cancer with immunotherapy, wherein the subject has been determined to have a higher expressed frameshift indel mutational burden in comparison to a reference sample.

7. A method of treating or preventing cancer in a subject which comprises treating a subject with cancer with immunotherapy, which subject has been identified as suitable for treatment with an immunotherapy by the method according to claim 1 or 2.

8. An immunotherapy for use in a method of treatment or prevention of cancer in a subject, the method comprising:
(i) identifying a subject with cancer who is suitable for treatment with immunotherapy by the method according to any of claim 1 or 2; and
(ii) treating said subject with an immunotherapy.

9. An immunotherapy for use in treating or preventing cancer in a subject, wherein the subject has been determined to have a higher expressed frameshift indel mutational burden in comparison to a reference sample.

10. An immunotherapy for use in treating or preventing cancer in a subject, which subject has been identified as suitable for treatment with immunotherapy by the method according to claim 1 or 2.

11. The method or immunotherapy for use according to any preceding claim wherein the expressed frameshift indel mutations are tumour suppressor gene expressed frameshift indel mutations.

12. The method or immunotherapy for use according to any preceding claim wherein the expressed frameshift indel mutations encode clonal neo-antigens.

13. The method or immunotherapy for use according to any preceding claim wherein the indel mutations are identified by Exome sequencing, RNA-seq, whole genome sequencing and/or targeted gene panel sequencing.

14. The method or immunotherapy for use according to any preceding claim wherein the sample is a tumour, blood or tissue sample from the subject.

15. The method or immunotherapy for use according to any preceding claim wherein the immunotherapy is immune checkpoint intervention or cell therapy.

16. The method or immune checkpoint intervention for use according to claim 15 wherein the immune checkpoint intervention interacts with CTLA4, PD-1, PD-L1, Lag-3, Tim-3, TIGIT or BTLA.

17. The method or immune checkpoint intervention for use according to claim 16 or claim 17 wherein the immune checkpoint intervention is pembrolizumab, nivolumab, atezolizumab or ipilimumab.

18. The method or immunotherapy for use according to claim 15 wherein said cell therapy is T cell therapy.

19. The method or immunotherapy for use according to any preceding claim wherein the cancer is selected from bladder cancer, gastric cancer, oesophageal cancer, breast cancer, colorectal cancer, cervical cancer, ovarian cancer, endometrial cancer, kidney cancer (renal cell), lung cancer (small cell, non-small cell and mesothelioma), brain cancer (gliomas, astrocytomas, glioblastomas), melanoma, merkel cell carcinoma, clear cell renal cell carcinoma (ccRCC), lymphoma, small bowel cancers (duodenal and jejunal), leukemia, pancreatic cancer, hepatobiliary tumours, germ cell cancers, prostate cancer, head and neck cancers, thyroid cancer and sarcomas.

20. The method or immunotherapy for use according to claim 19 wherein the cancer is selected from melanoma, Merkel cell carcinoma, renal cancer, non-small cell lung cancer (NSCLC), urothelial carcinoma of the bladder (BLAC), head and neck squamous cell carcinoma (HNSC), and MSI-high cancers

21. The method or immunotherapy for use according to claim 19 wherein the cancer is melanoma.

22. The method or immunotherapy for use according to claim 19 wherein the cancer is kidney cancer (renal cell).

23. The method or immunotherapy for use according to any preceding claim wherein the subject is a mammal, preferably a human, cat, dog, horse, donkey, sheep, goat, pig, cow, mouse, rat, rabbit or guinea pig.

24. The method or immunotherapy for use according to claim 22 wherein the subject is a human.
